SEQUENCE LISTING (19 sequences)

SEQ ID NO: 1 - protein, 183 aa, Streptomyces avidinii
MRKIVVAAIAVSLTTVSITASASADPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 2 - protein, 127 aa, Streptomyces avidinii
AEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 3 - protein, 127 aa, Artificial Sequence
  Synthetic Peptide
AEAGITGTWYAQLGDTFIVTAGADGALTGTYEAAVGNAESRYVLTGRYDSAPATDGSGTALGWTVAWKNNYRNAHSATTWSGQYVGGAEARINTQWLLTSGTTEANAWKSTLVGHDTFTKVKPSAAS

SEQ ID NO: 4 - protein, 133 aa, Streptomyces avidinii
  MISC_FEATURE (128)..(133): Poly-Histidine Tag
AEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASHHHHHH

SEQ ID NO: 5 - protein, 127 aa, Artificial Sequence
  Synthetic Peptide
AEAGITGTWYNQLGSTFIVTAGADGALTGTYESAVGNAESRYVLTGRYDSAPATDGSGTALGWTVAWKNNYRNAHSAITWSGQYVGGAEARINTQWLLTSGTTEANAWKSTLVGHDTFTKVKPSAAS

SEQ ID NO: 6 - DNA, 41 bp, Artificial Sequence
  Synthetic Oligonucleotide
ggcacctggtacgcccagctgggagacaccttcatcgttac

SEQ ID NO: 7 - DNA, 41 bp, Artificial Sequence
  Synthetic Oligonucleotide
gtaacgatgaaggtgtctcccagctgggcgtaccaggtgcc

SEQ ID NO: 8 - DNA, 41 bp, Artificial Sequence
  Synthetic Oligonucleotide
tctgaccggtacctacgaagccgctgttggtaacgctgaat

SEQ ID NO: 9 - DNA, 41 bp, Artificial Sequence
  Synthetic Oligonucleotide
attcagcgttaccaacagcggcttcgtaggtaccggtcaga

SEQ ID NO: 10 - DNA, 29 bp, Artificial Sequence
  Synthetic Oligonucleotide
cgctcactccgctatcacctggtctggcc

SEQ ID NO: 11 - DNA, 29 bp, Artificial Sequence
  Synthetic Oligonucleotide
ggccagaccaggtgatagcggagtgagcg

SEQ ID NO: 12 - DNA, 28 bp, Artificial Sequence
  Synthetic Oligonucleotide
tccagaattcgtaactaactaaaggaga

SEQ ID NO: 13 - DNA, 52 bp, Artificial Sequence
  Synthetic Oligonucleotide
agacaagcttttattaatggtggtgatggtgatgggaagcagcggacggttt

SEQ ID NO: 14 - DNA, 52 bp, Artificial Sequence
  Synthetic Oligonucleotide
ccggtcggcctgaacgatatcttcgaggcccagaagatcgagtggcacgaga

SEQ ID NO: 15 - DNA, 52 bp, Artificial Sequence
  Synthetic Oligonucleotide
gatctctcgtgccactcgatcttctgggcctcgaagatatcgttcaggccga

SEQ ID NO: 16 - protein, 152 aa, Gallus gallus
MVHATSPLLLLLLLSLALVAPGLSARKCSLTGKWTNDLGSNMTIGAVNSRGEFTGTYITAVTATSNEIKESPLHGTQNTINKRTQPTFGFTVNWKFSESTTVFTGQCFIDRNGKEVLKTMWLLRSSVNDIGDDWKATRVGINIFTRLRTQKE

SEQ ID NO: 17 - protein, 128 aa, Gallus gallus
ARKCSLTGKWTNDLGSNMTIGAVNSRGEFTGTYITAVTATSNEIKESPLHGTQNTINKRTQPTFGFTVNWKFSESTTVFTGQCFIDRNGKEVLKTMWLLRSSVNDIGDDWKATRVGINIFTRLRTQKE

SEQ ID NO: 18 - protein, 128 aa, Artificial Sequence
  Synthetic Peptide
ARKCSLTGKWTADLGDNMTIGAVNSRGEFTGTYIAAVTATSNEIKESPLHGTQNTINKRTQPTFGFTVNWKFSESTTVFTGQCFIDRNGKEVLKTMWLLRSSVNDIGDDWKATRVGINIFTRLRTQKE

SEQ ID NO: 19 - protein, 134 aa, Artificial Sequence
  Synthetic Peptide
ARKCSLTGKWTNDLGSNMTIGAVNSRGEFTGTYITAVTATSNEIKESPLHGTQNTINKRTQPTFGFTVNWKFSESTTVFTGQCFIDRNGKEVLKTMWLLRSSVNDIGDDWKATRVGINIFTRLRTQKEHHHHHH

(19) United States
(12) Patent Application Publication
(10) Pub. No.: US 2007/0105162 A1
(43) Pub. Date: May 10, 2007

(54) Monovalent avidin and streptavidin compositions

(75) Inventors: Alice Y. Ting, Allston, MA (US); Mark R. Howarth, Cambridge, MA (US)

Correspondence Address: WOLF GREENFIELD & SACKS, PC, FEDERAL RESERVE PLAZA, 600 ATLANTIC AVENUE, BOSTON, MA, 02210-2206, US

(73) Assignee: Massachusetts Institute of Technology, Cambridge, MA (US)

(21) Appl. No.: 11/588,879

(22) Filed: Oct. 27, 2006

Related U.S. Application Data
(60) Provisional application No. 60/731,166, filed on Oct. 28, 2005.

(51) Int. Cl. C07K 14/705; G01N 33/53; C07H 21/04; C12P 21/06

(52) U.S. Cl. 435/7.5; 435/69.1; 435/320.1; 435/325; 530/350; 536/23.5

(57) ABSTRACT

The invention relates, in part, to monovalent avidin and streptavidin compositions. The invention also relates to methods of preparing and using monovalent avidin and streptavidin compositions. In some aspects of the invention, the compositions are monovalent avidin or monovalent streptavidin with a single femtomolar biotin-binding site.

### RELATED APPLICATION

This application claims priority under 35 U.S.C. §119 from U.S. provisional application Ser. No. 60/731,166, filed Oct. 28, 2005, the contents of which is incorporated herein in its entirety.

### GOVERNMENT SUPPORT

This invention was made in part with government support under grant number P20GM072029-01 from the National Institutes of Health (NIH). The United States Government may have certain rights in this invention.

### FIELD OF THE INVENTION

The invention relates, in part, to monovalent avidin and streptavidin compositions and methods of preparing and using monovalent avidin and streptavidin compositions. The compositions include monovalent avidin and streptavidin with a single femtomolar biotin-binding site.

### BACKGROUND OF THE INVENTION

Streptavidin and avidin are used ubiquitously in biology because of the affinity and stability of their binding to biotin (Green, N. M. Methods in Enzymol. 184: 51-67, 1990). Streptavidin binds biotin with a femtomolar dissociation constant (Green, N. M. Methods in Enzymol. 184: 51-67, 1990). The tight and specific binding of avidin and streptavidin to biotin has led to the use of avidin and streptavidin for labeling and purification of biotinylated proteins, DNA and cells, for targeting of therapeutics proteins and drugs, for assembly of nanodevices. However, avidin and streptavidin are tetramers, which permits binding to multiple binding sites and can result in cross-linking of the bound molecules. The occurrence of cross-linking makes avidin and streptavidin tetramers unsuitable for many applications. Mutations that make the streptavidin protein monomeric do reduce cross-linking, but also reduce the biotin binding affinity by 10⁴ to 10⁵-fold (Qureshi, M. H. et al., J. Biol. Chem. 276: 46422-46428, 2001; Green, N. M. and Toms, J. C., Biochem. J. 133: 687-700, 1973; Laitinen, O. H. et al., J. Biol. Chem. 278: 4010-4014, 2003; Wu, S. C. and Wong, S. L., J. Biol. Chem. 280: 23225-23231, 2005), because part of the biotin binding site comes from a neighboring subunit (Chilkoti, A. et al., Proc. Natl. Acad. Sci. USA 92: 1754-1758, 1995; Sano, T and Cantor, C. R. Proc. Natl. Acad. Sci. USA 92: 3180-3184, 1995). Chemical treatment of avidin can make it monomeric and reduce biotin affinity from 10⁴ to 10⁵-fold. (Bayer, M. E. and Wilchek, M. Biochem J., 316 (pt1): 193-199, 1996). Avidin mutations that make it monomeric also reduce biotin affinity by 10⁴ to 10⁵-fold. (Laitinen, O. H. et al., J Biol Chem. 278(6): 4010-4014, 2003 Epub 2002; Laintinen, O. H. et al., J Biol Chem. 276(11): 8219-8224, 2001 Epub 2000).

Efforts have been made to reduce the multiple binding issues of streptavidin tetramers. Single mutations in the biotin binding site have been identified that reduce biotin binding affinity dramatically (Qureshi, M. H. et al., J Biol. Chem. 276: 46422-46428, 2001; Chilkoti, A. et al., Proc. Natl. Acad. Sci. USA 92: 1754-1758, 1995; Klumb, L. A. et al., Biochem. 37: 7657-7663, 1998), but these mutations can still leave K_{d} values in the nanomolar range and can disrupt tetramerization (Qureshi, M. H. et al., J Biol. Chem. 276: 46422-46428, 2001; Wu, S. C. and Wong, S. L., J. Biol. Chem. 280: 23225-23231, 2005). The double mutant N23A, S27D has one of the weakest reported affinities for biotin (K_{d} 7.1×10⁻⁵M) (Chen, I. and Ting, A. Y. Curr. Opin. Biotechnol. 16: 35-40, 2005) and is still a tetramer, but each monomer subunit can still bind biotinylated cells and thus the potential for cross-linking remains.

### SUMMARY OF THE INVENTION

The invention relates, in part, to compositions comprising a monovalent avidin and streptavidin tetramers, the production of the monovalent avidin and streptavidin tetramers, and their use in methods such as research, diagnostics, imaging, biomolecule labeling, single-particle tracking, nanotechnology, etc. The monovalent compositions of the invention are advantageous in that they tightly bind biotin and compounds comprising biotin but because only a single functional biotin binding site is present in each tetramer, cross-linking does not occur.

The invention also relates, in part, to compositions comprising avidin or streptavidin tetramers with 2 or 3 modified monomer subunits, with the remainder of the four subunits of the tetramer as wild-type avidin or streptavidin subunits, respectively. These chimeric avidin or streptavidin tetramers are useful when controlled multivalency is desired. The methods provided herein for producing such chimeric tetramers, and the multivalent tetramers of the invention may be useful for the construction of avidin-based or streptavidin-based conjugates with a defined number of binding sites for proteins fused to avidin-binding or streptavidin-binding peptides (Keefe, A. D. et al., Protein Expr. Purif. 23: 440-446, 2001; Lamla, T. and Erdmann, V. A. Protein Expr. Purif. 33: 39-47, 2004; Schmidt, T. G. and Skerra, A. J. Chromatogr. A 676: 337-345, 1994), or for DNA and RNA aptamers (Bittker, J. A. et al., Nat. Biotechnol. 20: 1024-1029, 2002; Srisawat, C. and Engelke, D. R. RNA 7: 632-641, 2001).

The invention also relates, in part, to additional avidin and streptavidin polymers and methods for their production and use. Avidin and streptavidin polymers may include two, three, or four avidin or streptavidin monomeric subunits, respectively, and include avidin or streptavidin dimers, trimers, and tetramers. Streptavidin does not form mixed tetramers with avidin.

According to one aspect of the invention, monovalent avidin tetramers are provided. The monovalent tetramers include three modified avidin monomer subunits and one wild-type avidin monomer subunit, wherein the modified avidin monomer subunits each bind biotin or a fragment thereof with a K_{d} of greater than or equal to about 1×10⁻⁴ M. In some embodiments, the wild-type avidin monomer subunit binds biotin or a fragment thereof, with a K_{d} of a wild-type avidin monomer subunit for binding biotin or a fragment thereof. In some embodiments, the monovalent avidin tetramer has a proximal avidin K_{d} for binding biotin or a fragment thereof. In some embodiments, the monovalent avidin tetramer has a single femtomolar biotin binding site. In some embodiments, the monovalent avidin tetramer has a proximal avidin overall biotin off rate. In some embodiments, the amino acid sequence of the modified avidin monomer subunit consists of the amino acid sequence of a wild-type avidin monomer subunit with at least three substituted amino acid residues. In some embodiments, the substituted amino acid residues are in the sequence of a biotin binding pocket of the avidin monomer subunit. In some embodiments, the three substituted amino acid residues are N12A, S16D, and T35A. In some embodiments, one or more of the modified or wild-type avidin monomer subunits includes a purification tag. In some embodiments, the purification tag is a polyhistidine tag. In some embodiments, the avidin monomer subunit that has the purification tag is the wild-type avidin monomer subunit. In some embodiments, the monovalent avidin tetramer includes a detectable label. In some embodiments, the monovalent avidin tetramer is made by mixing together avidin monomers under conditions in which the monomers associate into tetramers. In some embodiments, the monovalent avidin tetramer is made using a single-chain tetramer production method.

According to another aspect of the invention, monovalent avidin tetramers are provided. The monovalent avidin tetramers include three modified avidin monomer subunits and one wild-type avidin monomer subunit, wherein the wild-type avidin monomer subunit binds biotin or a fragment thereof with a K_{d} of a wild-type avidin monomer binding biotin or a fragment thereof. In some embodiments, the modified avidin monomer subunits bind biotin or fragment thereof with a K_{d} of greater than or equal to about 1×10⁻⁴ M. In some embodiments, the monovalent avidin tetramer has a proximal avidin K_{d} for binding biotin or a fragment thereof. In some embodiments, the monovalent avidin tetramer has a single femtomolar biotin binding site. In some embodiments, the monovalent avidin tetramer has a proximal avidin overall biotin off rate. In some embodiments, the amino acid sequence of the modified avidin monomer subunit consists of the amino acid sequence of a wild-type avidin monomer subunit with at least three substituted amino acid residues. In some embodiments, the substituted amino acid residues are in the sequence of a biotin binding pocket of the avidin monomer subunit. In some embodiments, the three substituted amino acid residues are N12A, S16D, and T35A. In some embodiments, one or more of the modified or wild-type avidin monomer subunits includes a purification tag. In some embodiments, the purification tag is a polyhistidine tag. In some embodiments, the avidin monomer subunit that includes the purification tag is the wild-type avidin monomer subunit. In some embodiments, the monovalent avidin tetramer includes a detectable label. In some embodiments, the monovalent avidin tetramer is made by mixing together avidin monomers under conditions in which the monomers associate into tetramers. In some embodiments, the monovalent avidin tetramer is made using a single-chain tetramer production method.

According to yet another aspect of the invention, monovalent avidin tetramers are provided. The monovalent avidin tetramers include three modified avidin monomer subunits and one wild-type avidin monomer subunit, wherein the monovalent avidin tetramer has a proximal avidin K_{d} for binding biotin or a fragment thereof. In some embodiments, the wild-type avidin monomer subunit binds biotin or a fragment thereof, with a K_{d} of a wild-type avidin monomer subunit for biding biotin or a fragment thereof. In some embodiments, the modified avidin monomer subunits each bind biotin or a fragment thereof with a K_{d} of greater than or equal to about 1×10⁻⁴ M. In some embodiments, the monovalent avidin tetramer has a single femtomolar biotin binding site. In some embodiments, the monovalent avidin tetramer has a proximal avidin overall biotin off rate. In some embodiments, the amino acid sequence of the modified avidin monomer subunit consists of the amino acid sequence of a wild-type avidin monomer subunit with at least three substituted amino acid residues. In some embodiments, the substituted amino acid residues are in the sequence of a biotin binding pocket of the avidin monomer subunit. In some embodiments, the three substituted amino acid residues are N12A, S16D, and T35A. In some embodiments, one or more of the modified or wild-type avidin monomer subunits includes a purification tag. In some embodiments, the purification tag is a polyhistidine tag. In some embodiments, the avidin monomer subunit that includes the purification tag is the wild-type avidin monomer subunit. In some embodiments, the monovalent avidin tetramer includes a detectable label. In some embodiments, the monovalent avidin tetramer is made by mixing together avidin monomers under conditions in which the monomers associate into tetramers. In some embodiments, the monovalent avidin tetramer is made using a single-chain tetramer production method.

According to another aspect of the invention, avidin monomer subunits that include a modified wild-type avidin monomer amino acid sequence and binds biotin or a fragment thereof with a K_{d} of greater than or equal to about 1×10⁻⁴ M are provided. In some embodiments, the amino acid sequence of the modified avidin monomer subunit consists of the amino acid sequence of a wild-type avidin monomer subunit with at least three substituted amino acid residues. In certain embodiments, the substituted amino acid residues are in the sequence of a biotin binding pocket of the avidin monomer subunit. In some embodiments, the three substituted amino acid residues are N12A, S16D, and T35A. In some embodiments, the avidin monomer subunit is associated with three additional avidin monomer subunits in the form of an avidin tetramer. In some embodiments, the avidin monomer subunits of the avidin tetramer that are not an avidin monomer subunit, are unmodified wild-type avidin monomer subunits. In certain embodiments, the avidin monomer subunit includes a purification tag. In certain embodiments, the avidin monomer subunit includes a detectable label.

According to yet another aspect of the invention, methods of making a plurality of monovalent avidin tetramers are provided. The methods include forming the tetramers by associating wild-type avidin monomer subunits and modified avidin monomer subunits, wherein the monovalent avidin tetramer has one or more of the following characteristics: (a) a proximal avidin K_{d} for binding biotin or a fragment thereof, (b) a single femtomolar biotin binding site, (c) a proximal avidin overall biotin off rate, wherein an avidin monomer subunit modification consists at least of the substituted amino acid residues N12A, S16D, and T35A in the amino acid sequence of the avidin monomer subunit and the unmodified avidin monomer subunit is a wild-type avidin monomer subunit. In some embodiments, the monovalent avidin tetramer includes a purification tag permitting monitoring of avidin monomer subunit association into tetramers having specific stoichiometric ratios of modified and unmodified avidin monomer subunits. In some embodiments, the purification tag is a polyhistidine tag. In some embodiments, the purification tag is attached to the unmodified avidin monomer subunit. In certain embodiments, the monovalent avidin tetramer includes a detectable label. In some embodiments, the wild-type and modified avidin monomers are associated by mixing avidin monomers under conditions under which the monomers associate into tetramers. In some embodiments, the wild-type and modified avidin monomers are associated using a single-chain tetramer production method.

According to another aspect of the invention, compositions that include monovalent avidin tetramers made by any embodiment of the aforementioned methods of making a plurality of monovalent avidin tetramers are provided.

According to yet another aspect of the invention, avidin tetramers that include N=1, 2, or 3 modified avidin monomer subunits and 4 minus N wild-type avidin monomer subunits, wherein: (a) each wild-type avidin monomer subunit binds biotin or a fragment thereof with a K_{d} of a wild-type avidin monomer subunit for binding biotin or a fragment thereof, (b) each modified avidin monomer subunit binds biotin or a fragment thereof with a K_{d} of greater than or equal to about 1×10⁻⁴ M, and (c) the avidin tetramer has a proximal avidin K_{d} for binding biotin or a fragment thereof, are provided. In certain embodiments, the avidin tetramer has 4 minus N femtomolar biotin binding sites. In some embodiments, the tetramer has a proximal avidin overall biotin off rate. In some embodiments, the sequence of the modified avidin monomer subunit consists of the sequence of a wild-type avidin monomer subunit with at least three substituted amino acid residues. In some embodiments, the substituted amino acid residues are in the sequence of a biotin binding pocket of the avidin monomer subunit. In certain embodiments, three of the three or more substituted amino acid residues are N12A, S16D, and T35A. In some embodiments, one or more of the avidin monomer subunits includes a purification tag. In some embodiments, the purification tag is a polyhistidine tag. In certain embodiments, the avidin monomer subunit that includes the purification tag is the wild-type avidin monomer subunit. In some embodiments, one or more of the avidin monomer subunits includes a detectable label. In some embodiments, the ratio of modified and unmodified avidin monomer subunits in the avidin tetramer is 3:1, 2:2, or 1:3. In some embodiments, the avidin tetramers are made by mixing wild-type and modified avidin monomers under conditions under which the monomers associate into tetramers. In certain embodiments, the tetramers are made using a single-chain tetramer production method. In some embodiments, a plurality of the aforementioned avidin tetramers are made that have a ratio of wild-type avidin monomer subunits to modified avidin monomer subunits of 1:3, 2:2, 3:1, or a mixture thereof.

According to another aspect of the invention, methods of making a plurality of avidin tetramers comprising 1, 2, or 3 modified avidin monomer subunits, wherein the tetramer is formed by associating wild-type avidin monomers with modified avidin monomers, wherein the avidin tetramers have one or more of the following characteristics: (a) each wild-type avidin monomer subunit binds biotin or a fragment thereof a with a K_{d} of a wild-type avidin monomer subunit for binding biotin or a fragment thereof, (b) the modified avidin monomer subunits bind biotin or a fragment thereof with. a K_{d} of greater than or equal to about 1×10⁻⁴ M, and (c) the avidin tetramer has a proximal avidin K_{d} for binding biotin or a fragment thereof, and (d) the tetramer has a proximal avidin K_{d} for binding biotin or a fragment thereof, are provided. In some embodiments, each wild-type avidin monomer subunit comprises a femtomolar biotin binding site. In some embodiments, the tetramer has a proximal avidin overall biotin off rate. In some embodiments, the amino acid sequence of the modified avidin monomer subunit consists of the amino acid sequence of a wild-type avidin monomer subunit with at least three substituted amino acid residues. In certain embodiments, the substituted amino acid residues are in the sequence of a biotin binding pocket of the avidin monomer. In some embodiments, three of the three or more substituted amino acid residues are N12A, S16D, and T35A. In some embodiments, one or more of the modified and/or wild-type avidin monomer subunits include a purification tag. In some embodiments, the purification tag is a polyhistidine tag. In some embodiments, the avidin monomer subunit that includes the purification tag is the wild-type avidin monomer subunit. In certain embodiments, one or more of the avidin monomer subunits includes a detectable label. In some embodiments, the ratio of modified and unmodified avidin monomer subunits in the avidin tetramers is 3:1, 2:2, 1:3 or a mixture thereof. In some embodiments, the wild-type and modified avidin monomers are associated by mixing monomers under conditions under which the monomers associate into tetramers. In certain embodiments, the wild-type and modified avidin monomers are associated using a single-chain tetramer production method.

According to another aspect of the invention, compositions that include avidin tetramers made by any embodiment of the aforementioned methods of making avidin tetramers are provided.

According to yet another aspect of the invention, methods of binding biotin or a fragment thereof are provided. The methods include contacting a biological sample that includes biotin or a fragment thereof with any of the monovalent avidin tetramers of the aforementioned aspects of the invention or any monovalent avidin tetramer made by any of the aforementioned methods of the invention under conditions that permit binding of biotin or a fragment thereof with a monovalent avidin tetramer.

According to yet another aspect of the invention avidin dimer or trimer molecules that include at least one modified avidin monomer subunit and at least one wild-type avidin monomer subunit are provided. In some embodiments, the avidin dimer or trimer molecule has a proximal avidin K_{d} for binding biotin or a fragment thereof. In some embodiments, the avidin dimer or trimer molecule has a proximal avidin overall biotin off rate. In some embodiments, the amino acid sequence of the modified avidin monomer subunit consists of the amino acid sequence of a wild-type avidin monomer subunit with at least one, two, or three substituted amino acid residues. In certain embodiments, the avidin dimer or trimer molecule is a monovalent avidin dimer or trimer molecule. In some embodiments, the wild-type avidin monomer subunit binds biotin or a fragment thereof with a K_{d} of a wild-type avidin monomer subunit for biding biotin or a fragment thereof. In some embodiments, the modified avidin monomer subunits each bind biotin or a fragment thereof with a K_{d} of greater than or equal to about 1×10⁻⁴ M. In certain embodiments, the monovalent avidin tetramer has a single femtomolar biotin binding site. In some embodiments, the amino acid sequence of the modified avidin monomer subunit consists of the amino acid sequence of a wild-type avidin monomer subunit with at least three substituted amino acid residues. In some embodiments, the substituted amino acid residues are in the sequence of a biotin binding pocket of the avidin monomer subunit. In certain embodiments, the three substituted amino acid residues are N12A, S16D, and T35A. In some embodiments, one or more of the modified or wild-type avidin monomer subunits includes a purification tag. In some embodiments, the purification tag is a polyhistidine tag. In some embodiments, the avidin monomer subunit that includes the purification tag is the wild-type avidin monomer subunit. In certain embodiments, the avidin dimer or trimer molecule includes a detectable label. In some embodiments, the avidin dimer or trimer is made using a single-chain production method.

According to yet another aspect of the invention streptavidin dimer or trimer molecules that include at least one modified streptavidin monomer subunit and at least one wild-type streptavidin monomer subunit are provided. In some embodiments, the streptavidin dimer or trimer molecule has a proximal streptavidin K_{d} for binding biotin or a fragment thereof. In some embodiments, the streptavidin dimer or trimer molecule has a proximal streptavidin overall biotin off rate. In some embodiments, the amino acid sequence of the modified streptavidin monomer subunit consists of the amino acid sequence of a wild-type streptavidin monomer subunit with at least one, two, or three substituted amino acid residues. In certain embodiments, the streptavidin dimer or trimer molecule is a monovalent streptavidin dimer or trimer molecule. In some embodiments, the wild-type streptavidin monomer subunit binds biotin or a fragment thereof with a K_{d} of a wild-type streptavidin monomer subunit for biding biotin or a fragment thereof. In some embodiments, the modified streptavidin monomer subunits each bind biotin or a fragment thereof with a K_{d} of greater than or equal to about 1×10⁻⁴ M. In certain embodiments, the monovalent streptavidin tetramer has a single femtomolar biotin binding site. In some embodiments, the amino acid sequence of the modified streptavidin monomer subunit consists of the amino acid sequence of a wild-type streptavidin monomer subunit with at least three substituted amino acid residues. In some embodiments, the substituted amino acid residues are in the sequence of a biotin binding pocket of the streptavidin monomer subunit. In certain embodiments, the three substituted amino acid residues are N23A, S27D, and S45A. In some embodiments, one or more of the modified or wild-type streptavidin monomer subunits includes a purification tag. In some embodiments, the purification tag is a polyhistidine tag. In some embodiments, the streptavidin monomer subunit that includes the purification tag is the wild-type streptavidin monomer subunit. In certain embodiments, the streptavidin dimer or trimer molecule includes a detectable label. In some embodiments, the streptavidin dimer or trimer is made using a single-chain production method.

In some aspects, the invention includes the use of the foregoing tetramers, trimers, or dimers and compositions in the preparation of a medicament.

These and other objects of the invention will be described in further detail in connection with the detailed description of the invention.

### BRIEF DESCRIPTION OF THE DRAWINGS

FIG. 1 shows schematic diagrams and digitized images of SDS-PAGE gels summarizing the generation of monovalent streptavidin. FIG. 1A is a schematic diagram of monovalent streptavidin structure. Wild-type streptavidin is a tetramer with 4 biotin binding sites (B=biotin). Monovalent streptavidin is a tetramer with 3 inactive subunits (dark grey squares) and one subunit that binds biotin with wild-type affinity (light grey square). FIG. 1B is a schematic diagram depicting strategy for making monovalent streptavidin. Inactivated streptavidin subunits (D) and wild-type streptavidin subunits (A) in a 3:1 ratio were refolded from denaturant, giving a mix of streptavidin heterotetramers. Tetramers with a single His₆-tagged wild-type subunit were purified on a Ni-NTA column. FIG. 1C is a digitized image of an SDS-PAGE gel of chimeric streptavidins under non-denaturing conditions. Streptavidin with 4 dead subunits (D4), wild-type streptavidin with a His₆-tag (A4), the initial product of refolding of D and A in a 3:1 ratio (mix), and chimeric tetramers with one (A1D3), two (A2D2), or three (A3D1) biotin binding subunits were loaded without boiling onto an 8% SDS-PAGE gel, and visualized by Coomassie staining. FIG. 1D shows a digitized image of an SDS-PAGE gel of chimeric streptavidins under denaturing conditions. Chimeric streptavidins with 0-4 A subunits were run as in FIG. 1C, except the samples were boiled before loading, to break the tetramer into constituent monomers. The changing ratio of A and D subunits can be seen.

FIG. 2 shows digitized images of SDS-PAGE gels illustrating the stability of monovalent streptavidin. FIG. 2A shows a digitized image of an SDS-PAGE gel demonstrating the stability of monovalent streptavidin to subunit exchange. 5 μM A1D3 in PBS was incubated at 26° C. or 37° C. for 1 hour (hr), 1 day (d), or 1 week (wk) and rearranged tetramers were detected by 8% SDS-PAGE, by comparison to the initial product of refolding of D and A in a 3:1 ratio (mix). FIG. 2B shows an SDS-PAGE gel results indicating stability of tetramer to heat denaturation. 5 μM wild-type streptavidin or A1D3 in PBS was incubated at the indicated temperature (° C.) for 3 min and loaded onto a 16% SDS-PAGE gel.

FIG. 3 shows spectra of representative data collected on mass spectrometry of chimeric streptavidins. Spectra are shown±biotin for D4, A4, A1D3, and the initial product of refolding of D and A in a 3:1 ratio (Mix). Vertical lines indicate the predicted m/z for the 15+ charge state of the different tetramers without biotin. The 15+ charge state generally gave the sharpest peaks but the 14+ and 16+ peaks are also indicated. Horizontal arrows indicate the shift in m/z caused by binding of biotin. The number of biotin molecules bound is shown in parentheses.

FIG. 4 shows two graphs indicating the comparative K_{d} of the dead tetramer D4 and monovalent streptavidin A1D3. FIG. 4A shows the K_{d} value determination for D4. 24 μM D4 was incubated with increasing concentrations of ³H-biotin. After >20 hr, the amount of bound ³H-biotin was determined by precipitating D4. Means of triplicate measurement are shown±1 s.d. The measured K_{d} for D4 was 9.18±1.17×10−4M (s.e.m.). FIG. 4B shows the K_{d} value determined for A1D3. For this determination increasing concentrations of A1D3 were incubated with 20 nM ³H-biotin and 60 nM wild-type streptavidin. After >20 hr, A1D3 was separated with Ni-NTA agarose, and the amount of ³H-biotin bound to wild-type streptavidin in the supernatant was measured. From this value, the amount of ³H-biotin bound to A1D3 was deduced. Means of triplicate measurement are shown±1 s.d. Some error bars are too small to be visible. This gave a K_{d} for A1D3 of 4.94±0.65×10−14M (s.e.m.).

FIG. 5 shows three graphs illustrating the monovalent streptavidin off-rate. FIG. 5A shows comparative off-rates of Wild-type (♦), A1D3 (▪), S45A (X) or T90I (▴) streptavidin, where each species was added in excess to biotin-4-fluorescein to quench its fluorescence. Excess competing biotin was added and fluorescence increase was monitored as biotin-4-fluorescein dissociated from streptavidin. 100% represents complete dissociation of biotin-4-fluorescein. Means of triplicate measurement are shown +1 s.d. FIG. 5B shows a magnification of the 0-10% region of the y-axis from FIG. 5A, to illustrate the similar dissociation curves for wild-type streptavidin and A1D3. FIG. 5C shows results of a determination of off-rate of wild-type streptavidin (◯) and A1D3 (▴) from biotin. A1D3 or wild-type streptavidin were incubated with ³H-biotin. Excess cold biotin was then added. After varying times at 37° C., the amount of bound ³H-biotin was determined by precipitating streptavidin. Means of triplicate measurement are shown±1 s.d. The measured off-rates were 5.17±0.25×10⁻⁵s⁻¹ (s.e.m.) for wild-type streptavidin and 6.14±0.19×10⁻⁵s⁻¹ (s.e.m.) for A1D3.

### BRIEF DESCRIPTION OF THE SEQUENCES

SEQ ID NO:1 is wild-type streptavidin sequence
from Genbank Accession No. P22629:
MRKIVVAAIAVSLTTVSITASASADPSKDSKAQVSAAEAGITGTWYNQLG
STFIVTAGADGALTGTYESAVGNAESRYVLTGRYDSAPATDGSGTALGWT
VAWKNNYRNAHSATTWSGQYVGGAEARINTQWLLTSGTTEANAWKSTLVG
HDTFTKVKPSAASIDAAKKAGVNNGNPLDAVQQ
SEQ ID NO:2 is wild-type core streptavidin
protein:
AEAGITGTWYNQLGSTFIVTAGADGALTGTYESAVGNAESRYVLTGRYDS
APATDGSGTALGWTVAWKNNYRNAHSATTWSGQYVGGAEARINTQWLLTS
GTTEANAWKSTLVGHDTFTKVKPSAAS
SEQ ID NO:3 is modified streptavidin monomer Dead
(D), N23A, S27D, S45A:
AEAGITGTWYAQLGDTFIVTAGADGALTGTYEAAVGNAESRYVLTGRYDS
APATDGSGTALGWTVAWKNNYRNAHSATTWSGQYVGGAEARINTQWLLTS
GTTEANAWKSTLVGHDTFTKVKPSAAS
SEQ ID NO:4 is unmodified streptavidin monomer
Alive (A) with polyhistidine tag:
AEAGITGTWYNQLGSTFIVTAGADGALTGTYESAVGNAESRYVLTGRYDS
APATDGSGTALGWTVAWKNNYRNAHSATTWSGQYVGGAEARINTQWLLTS
GTTEANAWKSTLVGHDTFTKVKPSAASHHHHHH
SEQ ID NO:5 is modified streptavidin monomer,
T90I:
AEAGITGTWYNQLGSTFIVTAGADGALTGTYESAVGNAESRYVLTGRYDS
APATDGSGTALGWTVAWKNNYRNAHSAITWSGQYVGGAEARINTQWLLTS
GTTEANAWKSTLVGHDTFTKVKPSAAS
SEQ ID NO:6 is sequence to introduce N23A and
S27D:
5′-ggcacctggtacgcccagctgggagacaccttcatcgttac-3′.
SEQ ID NO:7 is reverse complement of SEQ ID NO:6
5′-gtaacgatgaaggtgtctcccagctgggcgtaccaggtgcc-3′.
SEQ ID NO:8 is sequence to introduce S45A:
5′-tctgaccggtacctacgaagccgctgttggtaacgctgaat-3′.
SEQ ID NO:9 is reverse complement of SEQ ID NO:8:
5′-attcagcgttaccaacagcggcttcgtaggtaccggtcaga-3′.
SEQ ID NO:10 is primer sequence to introduce T90I:
5′-cgctcactccgctatcacctggtctggcc-3′.
SEQ ID NO:11 is reverse complement of
SEQ ID NO:10:
5′-ggccagaccaggtgatagcggagtgagcg-3′.
SEQ ID NO:12 is forward primer sequence to add six
histidine residues at the C-terminus of
streptavidin sequence
5′-tccagaattcgtaactaactaaaggaga-3′
SEQ ID NO: 13 is reverse primer sequence to add six
histidine residues at the C-terminus of
streptavidin:
5′-agacaagcttttattaatggtggtgatggtgatgggaagcagcggac
ggttt-3′
SEQ ID NO:14:
5′-ccggtcggcctgaacgatatcttcgaggcccagaagatcgagtggca
cgaga-3′
SEQ ID NO:15:
5′-gatctctcgtgccactcgatcttctgggcctcgaagatatcgttcag
gccga-3′
SEQ ID NO:16 is wild-type avidin sequence from
Genbank Accession No. X05343:
MVHATSPLLLLLLLSLALVAPGLSARKCSLTGKWTNDLGSNMTIGAVNSR
GEFTGTYITAVTATSNEIKESPLHGTQNTINKRTQPTFGFTVNWKFSEST
TVFTGQCFIDRNGKEVLKTMWLLRSSVNDIGDDWKATRVGINIFTRLRTQ
KE
SEQ ID NO:17 is wild-type core avidin protein
sequence:
ARKCSLTGKWTNDLGSNMTIGAVNSRGEFTGTYITAVTATSNEIKESPLH
GTQNTINKRTQPTFGFTVNWKFSESTTVFTGQCFIDRNGKEVLKTMWLLR
SSVNDIGDDWKATRVGINIFTRLRTQKE
SEQ ID NO:18 is modified avidin monomer Dead (D),
N12A, S16D, T35A:
ARKCSLTGKWTADLGDNMTIGAVNSRGEFTGTYIAAVTATSNEIKESPLH
GTQNTINKRTQPTFGFTVNWKFSESTTVFTGQCFIDRNGKEVLKTMWLLR
SSVNDIGDDWKATRVGINIFTRLRTQKE
SEQ ID NO:19 is unmodified avidin monomer Alive
(A) with polyhistidine tag:
ARKCSLTGKWTNDLGSNMTIGAVNSRGEFTGTYITAVTATSNEIKESPLH
GTQNTINKRTQPTFGFTVNWKFSESTTVFTGQCFIDRNGKEVLKTMWLLR
SSVNDIGDDWKATRVGINIFTRLRTQKEHHHHHH

### DETAILED DESCRIPTION OF THE INVENTION

Novel monovalent avidin tetramers have been developed that have a single femtomolar biotin-binding site that retains the binding affinity of a wild-type avidin tetramer. Monovalent avidin tetramers have been generated that containing three subunits that do not functionally bind biotin and one subunit with a wild-type biotin-binding pocket. The monovalent avidin tetramer that has been developed has similar affinity for biotin, off-rate, and thermostability to a wild-type avidin tetramer but is monovalent. Thus, monovalent avidin tetramers with only one functional biotin binding monomer subunit have been produced.

A monovalent avidin has been made that bound to biotin with an affinity and stability similar to wild-type avidin, but did not produce cross-linking. Monovalent avidin should enable one to make use of femtomolar binding affinity without additional unwanted multimerization. Other chimeric avidin tetramers A2D2 and A3D1, have also been purified for when controlled multivalency is desired. This approach may be useful for the construction of avidin-based conjugates with a defined number of binding sites for proteins fused to avidin-binding peptides, or for DNA and RNA aptamers. Given the remarkable range of uses to which avidin has been put, these avidins should be valuable building blocks for many new nano-architectures.

The invention disclosed herein describes novel monovalent avidin polymers and methods of making and using monovalent avidin polymers and methods of making and using modified avidin monomers. The discovery that a monovalent avidin can be prepared that has a femtomolar binding affinity for biotin and fragments thereof, facilitates the production and use of such a monovalent avidin in research applications; clinical applications including, but not limited to, diagnostics; imaging methods; pharmaceutical delivery, e.g. delivery of drugs, toxins; as well as other art-known methods that include the use of avidin tetramers. The invention relates to the production and use of various avidin monomers and avidin polymers. As used herein, the term “avidin polymer” means a avidin molecule that has two, three, or four avidin monomeric subunits. Avidin dimers, trimers, and tetramers have two, three, and four avidin monomeric subunits, respectively.

The binding capacity of an avidin polymer for biotin or a fragment thereof is referred to as its “valency”. A monovalent avidin polymer is an avidin tetramer that binds only a single biotin or fragment thereof. A multivalent avidin polymer has the capacity to bind to two, three, or four biotin molecules or fragments thereof. Thus, a wild-type avidin tetramer would be a polyvalent avidin molecule and could also be referred to as a tetravalent avidin polymer because it can bind four biotin molecules or fragments thereof.

The invention relates, in part, to the preparation of avidin polymers and the use of such avidin polymers to bind biotin and biotin conjugates. Various methods may be used to associate the avidin monomer subunits with each other to prepare avidin polymers. In one method, avidin monomer subunits are prepared and the monomer subunits are associated by mixing avidin monomer subunits together under conditions that permit four monomers to associate to form an avidin tetramer. In avidin molecules so prepared, the avidin monomer subunits are non-covalently linked together.

In another method of preparing an avidin tetramer, the nucleotide sequences that encode two, three, or four monomer avidin subunits are linked into a single gene, and the expression product of the single gene is an avidin polymer that includes the two, three, or four avidin monomers covalently linked together. This method is referred to herein as the “single-chain method” of producing an avidin polymer. Methods of single-chain production of avidin are described in Nordlund, H. R. et al., Biochem J. 2005 (Epub). Using the single-chain method a single polypeptide that includes a desired number and type of avidin subunits is expressed. In some embodiments, the desired avidin is an avidin dimer, which has only two avidin monomer subunits covalently linked to each other. In certain embodiments, the desired avidin is an avidin trimer, which has three avidin monomer subunits covalently linked together. In other embodiments, the desired avidin molecule is an avidin tetramer, which has four avidin monomer subunits covalently linked together. The terms “monomer”, “subunit”, and “monomer subunit” are used interchangeably herein.

A wild-type avidin tetramer includes four avidin wild-type monomers. A wild-type avidin monomer includes a single biotin binding site, also referred to herein as the biotin binding pocket, and is able to bind a single biotin molecule or fragment thereof. Thus, a wild-type avidin tetramer includes four biotin binding sites and is able to bind to four biotin molecules or fragments thereof. Avidin tetramers of the invention may include a combination of wild-type and modified avidin monomer subunits with the total number of subunits equal to four. Thus, avidin tetramers of the invention include tetramers with one, two, or three modified avidin monomers with the remaining monomers being wild-type monomers.

In some embodiments of the invention an avidin polymer is a dimer or trimer. An avidin dimer or trimer may include various combinations of wild-type and modified avidin monomer subunits. For example, an avidin dimer may be made up of one wild-type and one modified avidin monomer. An avidin trimer may have a ratio of wild-type avidin monomer to modified avidin monomer of 2:1, 1:2, 0:3, or 3:0.

The avidin monomers and/or polymers of the invention may be isolated monomers or tetramers. As used herein with respect to the monomers and polymers provided herein, “isolated” means separated from its native environment and present in sufficient quantity to permit its identification or use. Isolated, when referring to a protein or polypeptide, means, for example: (i) selectively produced by monomer association methods or single-chain production methods etc. or (ii) purified as by chromatography or electrophoresis. Isolated monomers or polymers of the invention may be, but need not be, substantially pure. Because an isolated monomer or polymer may be admixed with a pharmaceutically acceptable carrier in a pharmaceutical preparation, the polypeptide may comprise only a small percentage by weight of the preparation. The polypeptide is nonetheless isolated in that it has been separated from the substances with which it may be associated in production or living systems, i.e., isolated from other proteins, isolated from other types of monomers in the case of isolated monomers and isolated from other types of avidin polymers in the case of isolated avidin polymers (e.g. avidin dimers, trimers or tetramers). For example, a substantially pure avidin tetramer may be a tetramer that has a ratio of wild-type avidin monomers to modified avidin monomers of 1:3 that it is essentially free of avidin tetramers that have a ratio of wild-type avidin monomers to modified avidin monomers of 2:2 or 3:1. Substantially pure avidin monomers, dimers, trimers, and tetramers may be produced by using the methods provided herein or using other art-known techniques.

A plurality of avidin polymers of the invention may include avidin polymers with a single ratio of wild-type avidin monomers to modified avidin monomers (e.g. 1:3, 2:2, 3:1 or 1:1, etc) or may be a mixture of polymers that include two or more different ratios of wild-type avidin monomers to modified avidin monomers. For example, a plurality of avidin tetramers of the invention may include avidin tetramers with a single ratio of wild-type avidin monomers to modified avidin monomers (e.g. 1:3, 2:2, or 3:1) or may include a mixture of two or more different ratios of wild-type avidin monomers to modified avidin monomers.

Pluralities of avidin molecules of the invention, in some embodiments, include only monovalent avidin tetramers. In other embodiments, a plurality of avidin tetramers of the invention may include bivalent, trivalent, or tetravalent tetramers. If the avidin molecule is a dimer or trimer, a plurality may include only dimers or trimers. The dimers or trimers may be monovalent, divalent, or trivalent, depending on the whether the avidin molecule is a dimer or trimer. In some embodiments of the invention a plurality of avidin molecules may include mixtures of avidin molecules with different valences.

The avidin tetramers of the invention may be used in methods that include binding to biotin analogs. Examples of biotin analogs, although not intended to be limiting include: desthiobiotin, also known as dethiobiotin, selenobiotin, oxybiotin, homobiotin, norbiotin, iminobiotin, diaminobiotin, biotin sulfoxide, biotin sulfone, epibiotin, 5-hydroxybiotin, 2-thiobiotin, azabiotin, carbobiotin, and methylated derivatives of biotin, etc.

A wild-type avidin tetramer includes four wild-type monomer subunits, each of which binds biotin or a fragment thereof, with high affinity. The amino acid sequence of a wild-type avidin monomer subunit can be made from a precursor wild-type avidin protein that includes a core avidin sequence as well as a signal sequence. The complete sequence of wild-type avidin precursor protein is set forth as Genbank Accession No. X05343 and is referred to herein as SEQ ID NO:16. The skilled artisan will realize that conservative amino acid substitutions may be made in a wild-type avidin amino acid precursor or core sequence. As used herein, a “conservative amino acid substitution” refers to an amino acid substitution that does not alter the relative binding characteristics of the avidin monomer or tetramer for biotin or a fragment thereof, in which the amino acid substitution is made. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. A conservatively substituted wild-type avidin amino acid sequence will still be considered to be a wild-type avidin amino acid sequence as long as the avidin monomer (or polymer made up of such monomers) retains the wild-type ability and affinity to bind biotin or a fragment thereof. Thus, an avidin monomer, or tetramer made with a wild-type avidin amino acid sequence that includes one or more conservative amino acid substitutions, will retain the functional capabilities of a wild-type avidin monomer or tetramer and be referred to as a wild-type avidin monomer or tetramer.

With respect to the identification of specific amino acid residues in polypeptides and proteins of the invention, residues 1-24 of the avidin precursor protein sequence are removed by other bacterial proteases to yield the mature wild-type avidin. Residue 25 of the sequence set forth as Genbank Accession No. X05343 (SEQ ID NO:16) is considered to be residue one of the mature avidin. Residues 25-152 (with or without conservative amino acid substitutions) are considered to be the wild-type core avidin amino acid sequence. A wild-type core avidin amino acid sequence is set forth as SEQ ID NO:17. SEQ ID NO:19 also has a wild-type core avidin amino acid sequence and also has a polyhistidine purification tag attached. SEQ ID NO:18 is the sequence of a modified avidin monomer subunit.

As used herein, a the term “wild-type avidin subunit” means an avidin subunit that has a wild-type avidin monomer amino acid sequence. As used herein, the term “unmodified” when used with respect to an avidin subunit means that monomer subunit has the core amino acid sequence of a wild-type avidin monomer subunit. Thus, in an unmodified avidin monomer subunit, the core amino acid sequence is the same as the core wild-type avidin monomer amino acid sequence. An example of an unmodified wild-type avidin subunit is an Alive (A) type monomer subunit provided herein.

SEQ ID NO:17 has the core wild-type avidin monomer amino acid sequence. SEQ ID NO:19 also has the core wild-type avidin monomer subunit amino acid sequence and a polyhistidine tag sequence. Additions of residues or other compounds onto the core sequence, such as those for a tag or label, do not negate the unmodified status of an avidin monomer as long as the core wild-type avidin monomer amino acid sequence remains unchanged. Thus, SEQ ID NO:17 and SEQ ID NO:19 (with a polyhistidine tag attached) are both examples of unmodified avidin monomer sequences. Tagging and/or labeling sequences or compounds can be added to a core wild-type avidin monomer amino acid sequence and the monomer remains an unmodified avidin monomer as long as the core amino acid sequence of the avidin monomer subunit remains unchanged from that of the core wild-type avidin monomer sequence.

A modified avidin monomer has a modification of the core wild-type avidin amino acid sequence. A modification of a sequence of an avidin subunit is a change in the amino acid sequence of the avidin monomer subunit from the wild-type amino acid sequence. Modifications of an avidin amino acid sequence may include the substitution of one or more amino acid residues in the sequence for alternative amino acids. A substitution of one amino acid for another in the mature sequence of wild-type avidin (residues 25-152 of SEQ ID NO:16), which is also referred to as the core amino acid sequence of a wild-type avidin monomer subunit, is an example of a modification of an avidin subunit. As described above herein, residue 25 of the sequence set forth as Genbank Accession No. X05343 (SEQ ID NO:16) is considered to be residue one of mature wild-type avidin monomer. Using this numbering system the residues that are altered in the preparation of some modified monomers of the invention include residues N12, S16, and S35. An example of a modified avidin monomer subunit is a D subunit, which includes the following substitutions: N→A at position 12 in the amino acid sequence of mature wild-type avidin monomer; S→D at position 26 in the amino acid sequence of mature wild-type avidin monomer; and S→A at position 35 in the amino acid sequence of mature wild-type avidin monomer.

The sequence set forth as the Dead (D) monomer sequence includes the following substituted amino acid residues: N12A, S16D, and S35A (with the numbering based on the numbering of the mature avidin protein sequence). A Dead monomer sequence of the invention is set forth herein as SEQ ID NO:18. The sequence of the Alive (A) monomer subunit, as described above, has the unmodified core wild-type avidin monomer sequence and also may include a His₆ purification tag. The Alive avidin monomer subunit with a His₆ tag is set forth herein as SEQ ID NO:19. For use in some methods and preparations of the invention, the sequences set forth as SEQ ID NO:16, 17, and 18 are encoded in a plasmid with an initiating methionine, which is then removed by the E. coli. It will be understood that the presence of an initiating methionine is not an alteration of the core sequence thus is not a modification.

The invention includes in some aspects, a monovalent avidin tetramer. A monovalent avidin tetramer includes one wild-type avidin monomer subunit that maintains wild-type avidin binding affinity for biotin or fragments thereof, and three modified avidin subunits that have amino acid sequences that are modified from the wild-type avidin amino acid sequence. One type of modified avidin subunit used in a monovalent avidin tetramer of the invention is referred to herein as a Dead (D) type avidin monomer subunit and is a modified avidin monomer subunit. Other modified avidin subunits that may be used in monomers and monovalent tetramers, trimers, or dimers of the invention may have an amino acid sequence that includes more than three amino acid modifications of the sequence of a wild-type avidin. In some embodiments, a modified avidin subunit that has essentially no functional binding to biotin or a fragment thereof, has 4, 5, 6, or more modifications to the sequence of a wild type avidin. A preferred monovalent avidin tetramer of the invention includes wild-type and modified avidin subunits in a 1:3 ratio. A monovalent avidin tetramer of the invention contains only a single functional biotin binding subunit. In some preferred monovalent avidin tetramers, all three modified avidin monomer subunits have the same type of sequence modification. The wild-type avidin monomer subunit may also include a purification tag that may be used for the preparation and purification of monovalent avidin tetramers.

A wild-type avidin monomer subunit may, but need not, include a purification tag that may be used for the preparation and purification of monovalent avidin tetramers. An example of a methods for purification of a monovalent avidin tetramer that utilizes a purification tag is provided in the Examples section. An example of one alternative method of purifying a monovalent avidin tetramer without use of a purification tag includes separation of various tetramers with an iminobiotin column. With an iminobiotin column, D4 tetramers would not bind the column and other avidin tetramers would be eluted in the order A1D3, A2D2, A3D1, and then A4 with the later tetramers eluting with decreasing pH. Those of ordinary skill in the art will recognize that additional methods can also be used for separating and/or purifying avidin tetramers that have differing ratios of A to D avidin subunits.

Functional features of avidin polymers and monomers can be determined and are useful for characterizing polymers that include different combinations of wild-type and modified avidin monomer subunits. One such functional feature is a the binding affinity for biotin or a fragment thereof of an avidin monomer or polymer. As is recognized in the art, binding affinity can be expressed in terms of the dissociation of bound biotin or a fragment thereof from the avidin monomer or polymer to which it is bound. Thus, binding affinity can be expressed as the dissociation constant (K_{d}) of binding of an avidin monomer or polymer for biotin or a fragment thereof. The binding affinity of an avidin monomer or polymer can be determined as described below herein, or using other art-known methods. The binding affinity of an avidin monomer or polymer of the invention can be compared to the binding affinity of a wild-type avidin monomer or polymer determined under substantially identical conditions. For example, if wild-type avidin tetramer is determined to have a K_{d} of about 1×10⁻¹⁵ M when binding biotin or a fragment thereof, an avidin tetramer of the invention can be tested under the same conditions to allow a comparison of the affinity of the avidin tetramer of the invention to that of a wild-type avidin tetramer.

In some instances, it may be desirable to have avidin monomers that have reduced level of biotin binding affinity (as compared to wild-type) and to use such a modified avidin monomer as a subunit of an avidin tetramer. Modified avidin monomers are provided herein that have a binding affinity significantly lower than wild-type avidin monomer biotin binding affinity. A Dead (D) monomer subunit of the invention has much reduced or no functional binding to biotin or a fragment thereof. A dead (D) avidin monomer subunit may have a K_{d} of about 1 mM. In some embodiments, the K_{d} of a Dead (D) avidin subunit is greater than or equal to 1×10⁻¹ M. In some embodiments, the K_{d} of a Dead (D) avidin subunit is greater than or equal to 1×10⁻²M. some embodiments, the K_{d} of a Dead (D) avidin subunit is greater than or equal to 5×10⁻³ M (5 mM). In some embodiments, the K_{d} of a Dead (D) avidin subunit is greater than or equal to 1×10⁻³ M (1 mM). In certain embodiments, the K_{d} of a Dead (D) avidin subunit is greater than or equal to 5×10⁻⁴ M. In certain embodiments, the K_{d} of a Dead (D) avidin subunit is greater than or equal to 1×10⁻⁴ M. In certain embodiments, the K_{d} of a Dead (D) avidin subunit is greater than or equal to 5×10⁻⁵ M. In certain embodiments, the K_{d} of a Dead (D) avidin subunit is greater than or equal to 1×10⁻⁵ M. In some embodiments of the invention, the affinity of the Dead (D) avidin subunit is so low as to result in essentially no functional binding of the Dead (D) avidin monomer subunit to biotin or a fragment thereof. Thus, an avidin tetramer of the invention that includes three Dead (D) avidin monomer subunits and one wild-type avidin subunit will bind biotin or a fragment thereof at the single wild-type biotin binding site, and thus is defined as a monovalent avidin tetramer.

A monovalent avidin polymer of the invention has a single biotin binding site and that has femtomolar binding affinity for biotin or a fragment thereof. As used herein, femtomolar avidin binding affinity means a K_{d} of from about 1×10⁻¹⁵ M to about 9.99×10⁻¹³ M for that monomer binding site. The Alive (A) subunit in a monovalent avidin tetramer of the invention may bind biotin or a fragment thereof with a wild-type avidin monomer subunit binding affinity.

A monovalent avidin tetramer of the invention may have a proximal avidin K_{d} for binding biotin or a fragment thereof. As used herein, the term “proximal avidin K_{d}” means having a K_{d} that is between 1×10⁻¹²M and the K_{d} of a wild-type avidin tetramer or up to 10-fold lower than the K_{d} for wild-type avidin tetramers. Thus, a proximal avidin K_{d} may be a level from about 1×10⁻¹² down through the K_{d} of wild-type avidin tetramer or below wild-type K_{d} to as low as about 1×10⁻¹⁶ M. Thus, a proximal avidin K_{d} may be 1×10⁻¹²M, 5×10⁻¹³M, 1×10⁻¹³M, 5×10⁻¹⁴ M or any level in between 1×10⁻¹²M and about 1×10⁻¹⁶M. In addition, for the generation of monovalent avidin, affinity changes may be determined by assessing the affinity of the monovalent avidin to a biotin surrogate, such as iminobiotin, or to a biotin conjugate because the binding affinity for avidin to biotin is so high that it is difficult to measure that affinity of avidin to biotin.

A second functional feature of avidin tetramer and monomer binding that can be determined and may be useful to assess various tetramers and monomers of the invention is the off-rate of biotin from avidin after binding. Off-rate is a measure of time it takes for biotin to dissociate from an avidin monomer or polymer to which it has bound. A faster off-rate indicates less stable binding than an avidin monomer or tetramer with a slower off-rate, which has more stable binding between the biotin and the avidin monomer or tetramer, respectively. A determination of the off-rate of biotin binding to a avidin monomer or tetramer thus can provide information regarding the stability of the binding. Off-rate determinations can be made using methods provided in the Examples section as well as using as additional art-known methods of measuring binding dissociation. An avidin monomer or tetramer of the invention may have a proximal avidin overall biotin off rate. As used herein, the term “proximal avidin overall biotin off rate” means that the percentage of biotin dissociation from the avidin monomer or tetramer is no more than 1%, 5%, 10%, 15%, 20%, or 25% higher (including all intervening percentages) than the percentage of biotin dissociation from a wild-type avidin monomer or tetramer, respectively, under substantially identical conditions. For example, the biotin off rate of an avidin tetramer of the invention and a wild-type avidin tetramer can be determined under the essentially the same conditions and the percent dissociation of biotin from the avidin tetramer will be no more than 1%, 5%, 10%, 15%, 20%, or 25% higher (including all intervening percentages) than the percentage of biotin dissociation from a wild-type avidin tetramer.

There are also functional features of avidin polymers that may be useful to assess various avidin polymers of the invention. One functional aspect that is useful to assess avidin polymers is the stability of avidin polymers over time. The stability of avidin tetramers involves a determination of whether a polymer would rearrange its subunits over time. A rearrangement of subunits may include a change in the ratio of different types of monomers in a polymer. For example, in a plurality of avidin tetramers that have a 3:1 ratio of a modified avidin monomer to wild-type avidin monomers, the ratio of subunit types may change over time, thus resulting in a mixed population of 3:1, 2:2, and 1:3 ratios of modified to wild-type avidin subunits. Low levels of stability result in faster shifts in avidin monomer ratios in an avidin polymer and higher levels of stability result in reduced changes in the ratio of avidin subunit types in an avidin polymer.

The thermostability of avidin polymers is another functional feature that may be used to assess avidin polymers of the invention. Thermal stability can be assessed by heating avidin polymers and separation of monomers from tetramers on polyacrylamide gels, as a determination of whether the avidin monomers remain associated in the polymers or dissociate. In some embodiments, dissociation of monomeric subunits of polymers means dissociation into monomeric subunits. In other embodiments, it may mean loss of one or more monomer subunits with dimer or trimer polymers remaining. Methods for determining thermostability of an avidin polymer are provided herein and also include additional assessment methods known in the art.

The modified and wild-type monomers and the monovalent and polyvalent avidin polymers of the invention may include a tag or label. In some embodiments, a tag is a purification tag. Purification tags of the invention include, but are not limited to polyhistidine tags (e.g. a His₆ tag). Additional types of purification tag sequences are known in the art and may be used in conjunction with the avidin polymers of the invention. Examples of purification tags, although not intended to be limiting, include the HQ tag from Promega (Madison, Wis.) that has a sequence of HQHQHQ, a FLAG tag (DYKDDDDK), or numerous other epitope tags known in the art. See, for example, Jarvik, J. W. and Telmer, C. A. Annu. Rev. Genet. 32: 601-618, 1998.

In some embodiments of the invention, an avidin monomer subunit or avidin polymer of the invention is linked to a detectable label. Detectable labels useful in the invention include, but are not limited to: a fluorescent label, an enzyme label, a radioactive label, visual label (e.g. a metallic label such as ferritin or gold), a nuclear magnetic resonance active label, an electron spin resonance label, a positron emission tomography label, a luminescent label, and a chromophore label. The detectable labels of the invention can be attached to the avidin monomer subunits or avidin polymers of the invention by standard protocols known in the art. In some embodiments, the detectable labels may be covalently attached to an avidin monomer or polymer of the invention. The covalent binding can be achieved either by direct condensation of existing side chains or by the incorporation of external bridging molecules. In some embodiments a detectable label may be attached to an avidin monomer or polymer of the invention using genetic methods. In some embodiments, a label may be attached by conjugating a moiety of interest (e.g. the labeling moiety) to biotin or a biotin analog and then non-covalent binding to the avidin tetramer. In some embodiments of the invention, more than one type of detectable label may be attached to an avidin monomer or polymer of the invention.

The avidin monomers and polymers of the invention bind to biotin or fragments thereof. By biotin fragments is meant a fragment of biotin that is sufficiently unchanged from the structure of biotin to be recognized and bound by an avidin monomer or polymer of the invention and or by a wild-type monomer or polymer. The biotin fragments may be considered to be functional biotin fragments. By “functional” biotin fragments, is meant that the biotin fragment is recognized by and can be bound by an avidin monomer or polymer of the invention.

The invention includes the use of an avidin monomer or polymer of the invention to bind to a biotin molecule or fragment thereof that is conjugated to an additional molecule or compound. Examples of such molecules or compounds, though not intended to be limiting, include proteins, nucleic acids, fatty acids, carbohydrates, small molecules, enzymes, antibodies, drug molecules, chemical compounds, cells, etc. Biotin is extensively used by those of skill in the scientific arts in labeling and tracking methods. Therefore, one of ordinary skill the art will recognize that the avidin molecules of the invention may be used to bind numerous different types of biotin conjugates. In some embodiments, a biotin or biotin molecule (conjugated or not) may be in solution or may be attached to a surface. Examples of surfaces to which an avidin polymer of the invention may be attached include, but are not limited to, a magnetic or chromatographic bead or particle bead or a chromatography support or other support.

The avidin monomers and polymers of the invention can be used for a wide variety of purposes including, but not limited to: cell sorting, cell labeling, drug delivery, imaging methods, etc. The avidin polymers of the invention can be linked to labels, delivery molecules, cells, etc for use in various technologies. The avidin polymers of the invention are also useful for imaging, including real-time imaging in vitro and in vivo. For some uses, avidin may be favored over streptavidin for in vivo applications because avidin is less immunogenic than streptavidin. There are many versions of avidin commonly available that have reduced non-specific binding, and are suitable for use in a number of research and other applications. One such example of an avidin version is Neutravidin. For review, see: Airenne, K. J. et al., Biomol. Eng. 16(1-4): 87-92, 1999.

An example of the use of an avidin polymer of the invention, though not intended to be limiting, is the use of the avidin polymer to isolate a target molecule or compound from a complex mixture or solution. In such embodiments, an avidin polymer of the invention can be attached to a targeting molecule and contacted with the complex mixture. The targeting molecule, attached to the avidin polymer, binds to the target and the complex mixture can be contacted with biotin or a fragment thereof, either alone, or in a conjugated form. Binding of the avidin polymer of the invention to the biotin or fragment thereof enables detection of the bound target in the complex mixture. Additionally, standard separation methods can then be used to separate the bound target molecule or compound from the complex mixture.

Another example of methods in which the avidin polymers of the invention, including the monovalent avidin tetramers described herein, may be used is in single-particle tracking, which is described in the Examples section herein. Additional labeling, imaging, cell sorting, and delivery methods for which the avidin polymers of the invention may be used, include a wide variety of art-known methods that include the use of avidin/biotin interactions.

Additional uses for avidin monomers and polymers of the invention may also include control of assembly of nanodevices. For example, binding of controlled numbers of biotinylated DNA, biotinylated proteins or biotinylated inorganic particles (including carbon nanotubes and quantum dots) to a surface or bead for systems detecting biological analytes or for building electrical circuits. An example of an application is the targeting to chemically biotinylated erythrocytes of avidin bound to drugs or other proteins. Erythrocytes may be used as drug delivery vehicles and may be useful because of their long circulation time.

Novel monovalent streptavidin tetramers have been developed that have a single femtomolar biotin-binding site that retains the binding affinity of a wild-type streptavidin tetramer. Monovalent streptavidin tetramers have been generated that containing three subunits that do not functionally bind biotin and one subunit with a wild-type biotin-binding pocket. The monovalent streptavidin tetramer that has been developed has similar affinity for biotin, off-rate, and thermostability to a wild-type streptavidin tetramer but is monovalent. Thus, monovalent streptavidin tetramers with only one functional biotin binding monomer subunit have been produced.

A monovalent streptavidin has been made that bound to biotin with an affinity and stability similar to wild-type streptavidin, but did not produce cross-linking. Monovalent streptavidin should enable one to make use of femtomolar binding affinity without additional unwanted multimerization. Other chimeric streptavidin tetramers A2D2 and A3D1, have also been purified for when controlled multivalency is desired. This approach may be useful for the construction of streptavidin-based conjugates with a defined number of binding sites for proteins fused to streptavidin-binding peptides (Keefe, A. D. et al., Protein Expr. Purif. 23: 440-446, 2001; Lamla, T. and Erdmann, V. A. Protein Expr. Purif. 33: 39-47, 2004; Schmidt, T. G. and Skerra, A. J. Chromatogr. A 676: 337-345, 1994), or for DNA and RNA aptamers (Bittker, J. A. et al., Nat. Biotechnol. 20: 1024-1029, 2002; Srisawat, C. and Engelke, D. R. RNA 7: 632-641, 2001). Given the remarkable range of uses to which streptavidin has been put, these streptavidins should be valuable building blocks for many new nano-architectures.

The invention disclosed herein describes novel monovalent streptavidin tetramers and methods of making and using monovalent streptavidin tetramers and methods of making and using modified streptavidin monomers. The discovery that a monovalent streptavidin can be prepared that has a femtomolar binding affinity for biotin and fragments thereof, facilitates the production and use of such a monovalent streptavidin in research applications; clinical applications including, but not limited to, diagnostics; imaging methods; pharmaceutical delivery, e.g. delivery of drugs, toxins; as well as other art-known methods that include the use of streptavidin tetramers. The invention relates to the production and use of various streptavidin monomers and streptavidin tetramers. The invention relates to the production and use of various streptavidin monomers and streptavidin polymers. As used herein, the term “streptavidin polymer” means a streptavidin molecule that has two, three, or four streptavidin monomeric subunits. Streptavidin dimers, trimers, and tetramers have two, three, and four streptavidin monomeric subunits, respectively.

The binding capacity of a streptavidin polymer for biotin or a fragment thereof is referred to as its “valency”. A monovalent streptavidin polymer is a streptavidin tetramer that binds only a single biotin or fragment thereof. A multivalent streptavidin polymer has the capacity to bind to two, three, or four biotin molecules or fragments thereof. Thus, a wild-type streptavidin tetramer would be a polyvalent streptavidin molecule and could also be referred to as a tetravalent streptavidin polymer because it can bind four biotin molecules or fragments thereof.

The invention relates, in part, to the preparation of streptavidin polymers and the use of such streptavidin polymers to bind biotin and biotin conjugates. Various methods may be used to associate the streptavidin monomer subunits with each other to prepare a streptavidin polymers. In one method, streptavidin monomer subunits are prepared and the monomer subunits are associated by mixing streptavidin monomer subunits together under conditions that permit four monomers to associate to form a streptavidin tetramer. In streptavidin molecules so prepared, the streptavidin monomer subunits are non-covalently linked together.

In another method of preparing a streptavidin tetramer, the nucleotide sequences that encode two, three, or four monomer streptavidin subunits are linked into a single gene, and the expression product of the single gene is a streptavidin polymer that includes the two, three, or four streptavidin monomers covalently linked together. This method is referred to herein as the “single-chain method” of producing a streptavidin polymer. Using the single-chain method a single polypeptide that includes a desired number and type of streptavidin subunits is expressed. In some embodiments, the desired streptavidin is a streptavidin dimer, which has only two streptavidin monomer subunits covalently linked to each other. In certain embodiments, the desired streptavidin is a streptavidin trimer, which has three streptavidin monomer subunits covalently linked together. In other embodiments, the desired streptavidin molecule is a streptavidin tetramer, which has four streptavidin monomer subunits covalently linked together. The terms “monomer”, “subunit”, and “monomer subunit” are used interchangeably herein.

A wild-type streptavidin tetramer includes four streptavidin wild-type monomers. A wild-type streptavidin monomer includes a single biotin binding site, also referred to herein as the biotin binding pocket, and is able to bind a single biotin molecule or fragment thereof. Thus, a wild-type streptavidin tetramer includes four biotin binding sites and is able to bind to four biotin molecules or fragments thereof. Streptavidin tetramers of the invention may include a combination of wild-type and modified streptavidin monomer subunits with the total number of subunits equal to four. Thus, streptavidin tetramers of the invention include tetramers with one, two, or three modified streptavidin monomers with the remaining monomers being wild-type monomers.

In some embodiments of the invention a streptavidin polymer is a dimer or trimer. A streptavidin dimer or trimer may include various combinations of wild-type and modified streptavidin monomer subunits. For example, a streptavidin dimer may be made up of one wild-type and one modified streptavidin monomer. A streptavidin trimer may have a ratio of wild-type streptavidin monomer to modified streptavidin monomer of 2:1, 1:2, 0:3, or 3:0.

The streptavidin monomers and/or polymers of the invention may be isolated monomers or tetramers. As used herein with respect to the monomers and polymers provided herein, “isolated” means separated from its native environment and present in sufficient quantity to permit its identification or use. Isolated, when referring to a protein or polypeptide, means, for example: (i) selectively produced by monomer association methods or single-chain production methods etc. or (ii) purified as by chromatography or electrophoresis. Isolated monomers or polymers of the invention may be, but need not be, substantially pure. Because an isolated monomer or polymer may be admixed with a pharmaceutically acceptable carrier in a pharmaceutical preparation, the polypeptide may comprise only a small percentage by weight of the preparation. The polypeptide is nonetheless isolated in that it has been separated from the substances with which it may be associated in production or living systems, i.e., isolated from other proteins, isolated from other types of monomers in the case of isolated monomers and isolated from other types of streptavidin polymers in the case of isolated streptavidin polymers (e.g. streptavidin dimers, trimers or tetramers). For example, a substantially pure streptavidin tetramer may be a tetramer that has a ratio of wild-type streptavidin monomers to modified streptavidin monomers of 1:3 that it is essentially free of streptavidin tetramers that have a ratio of wild-type streptavidin monomers to modified streptavidin monomers of 2:2 or 3:1. Substantially pure streptavidin monomers, dimers, trimers, and tetramers may be produced by using the methods provided herein or using other art-known techniques.

A plurality of streptavidin polymers of the invention may include streptavidin polymers with a single ratio of wild-type streptavidin monomers to modified streptavidin monomers (e.g. 1:3, 2:2, or 3:1, 1:1, 0:1, 1:0, etc) or may be a mixture of polymers that include two or more different ratios of wild-type streptavidin monomers to modified streptavidin monomers. For example, a plurality of streptavidin tetramers of the invention may include streptavidin tetramers with a single ratio of wild-type streptavidin monomers to modified streptavidin monomers (e.g. 1:3, 2:2, or 3:1) or may include a mixture of two or more different ratios of wild-type streptavidin monomers to modified streptavidin monomers.

Pluralities of streptavidin molecules of the invention, in some embodiments, include only monovalent streptavidin tetramers. In other embodiments, a plurality of streptavidin tetramers of the invention may include bivalent, trivalent, or tetravalent tetramers. If the streptavidin molecule is a dimer or trimer, a plurality may include only dimers or trimers. The dimers or trimers may be monovalent, divalent, or trivalent, depending on the whether the streptavidin molecule is a dimer or trimer. In some embodiments of the invention a plurality of streptavidin molecules may include mixtures of streptavidin molecules with different valences.

The streptavidin polymers of the invention may be used in methods that include binding to biotin analogs. Examples of biotin analogs, although not intended to be limiting include: desthiobiotin, also known as dethiobiotin, selenobiotin, oxybiotin, homobiotin, norbiotin, iminobiotin, diaminobiotin, biotin sulfoxide, biotin sulfone, epibiotin, 5-hydroxybiotin, 2-thiobiotin, azabiotin, carbobiotin, and methylated derivatives of biotin, etc.

A wild-type streptavidin tetramer includes four wild-type monomer subunits, each of which binds biotin or a fragment thereof, with high affinity. The amino acid sequence of a wild-type streptavidin monomer subunit can be made from a precursor wild-type streptavidin protein that includes a core streptavidin sequence as well as a signal sequence. The complete sequence of wild-type streptavidin precursor protein is set forth as Genbank Accession No. P22629 and is referred to herein as SEQ ID NO:1. The skilled artisan will realize that conservative amino acid substitutions may be made in a wild-type streptavidin amino acid precursor or core sequence. As used herein, a “conservative amino acid substitution” refers to an amino acid substitution that does not alter the relative binding characteristics of the streptavidin monomer or tetramer for biotin or a fragment thereof, in which the amino acid substitution is made. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. A conservatively substituted wild-type streptavidin amino acid sequence will still be considered to be a wild-type streptavidin amino acid sequence as long as the streptavidin monomer (or polymer made up of such monomers) retains the wild-type ability and affinity to bind biotin or a fragment thereof. Thus, a streptavidin monomer, or tetramer made with a wild-type streptavidin amino acid sequence that includes one or more conservative amino acid substitutions, will retain the functional capabilities of a wild-type streptavidin monomer or tetramer and be referred to as a wild-type streptavidin monomer or tetramer. Examples of conservative substitutions, although not intended to be limiting include E116A and T106A. (see Avantinis, S. K. et al., Chembiochem. 3(12): 1229-1234, 2002). Those of ordinary skill in the art will recognize additional conservative substitutions that do not negate the functional capability of wild-type streptavidin monomer or polymer.

With respect to the identification of specific amino acid residues in polypeptides and proteins of the invention, residues 1-24 of the precursor protein sequence are removed by bacterial proteases, to yield the mature wild-type streptavidin protein. Residue 25 of the sequence set forth as Genbank Accession No. P22629 (SEQ ID NO:1) is considered to be residue one of the mature streptavidin. Residues 37-163 of the sequence set forth as SEQ ID NO:1 (with or without conservative amino acid substitutions) are considered to be the wild-type core streptavidin amino acid sequence. A wild-type core streptavidin amino acid sequence is set forth as SEQ ID NO:2. SEQ ID NO:4 also has a wild-type core streptavidin amino acid sequence and also has a polyhistidine purification tag attached. SEQ ID NO:3 is the sequence of a modified streptavidin monomer subunit. Core streptavidin includes the sequence of streptavidin with terminal amino acids removed, thereby improving biotin conjugate binding and protein stability (see Sano, T. et al., J Biol Chem. 270(47): 28204-28209, 1995). The streptavidin tetramers of the invention may also be made using recombinant core streptavidin sequences.

As used herein, a the term “wild-type streptavidin subunit” means a streptavidin subunit that has a wild-type streptavidin monomer amino acid sequence. As used herein, the term “unmodified” when used with respect to a streptavidin subunit means that monomer subunit has the core amino acid sequence of a wild-type streptavidin monomer subunit. Thus, in an unmodified streptavidin monomer subunit, the core amino acid sequence is the same as the core wild-type streptavidin monomer amino acid sequence. An example of an unmodified wild-type streptavidin subunit is an Alive (A) type monomer subunit provided herein.

SEQ ID NO:2 has the core wild-type streptavidin monomer amino acid sequence. SEQ ID NO:4 also has the core wild-type streptavidin monomer subunit amino acid sequence and a polyhistidine tag sequence. Additions of residues or other compounds onto the core sequence, such as those for a tag or label, do not negate the unmodified status of a streptavidin monomer as long as the core wild-type streptavidin monomer amino acid sequence remains unchanged. Thus, SEQ ID NO:2 and SEQ ID NO:4 (with a polyhistidine tag attached) are both examples of unmodified streptavidin monomer sequences. Tagging and/or labeling sequences or compounds can be added to a core wild-type streptavidin monomer amino acid sequence and the monomer remains an unmodified streptavidin monomer as long as the core amino acid sequence of the streptavidin monomer subunit remains unchanged from that of the core wild-type streptavidin monomer sequence.

A modified streptavidin monomer has a modification of the core wild-type streptavidin amino acid sequence. A modification of a sequence of a streptavidin subunit is a change in the amino acid sequence of the streptavidin monomer subunit from the wild-type amino acid sequence. Modifications of a streptavidin amino acid sequence may include the substitution of one or more amino acid residues in the sequence for alternative amino acids. A substitution of one amino acid for another in the mature sequence of wild-type streptavidin (residues 25-163 of SEQ ID NO:1), is an example of a modification of a streptavidin subunit. As described above herein, residue 25 of the sequence set forth as Genbank Accession No. P22629 (SEQ ID NO:1) is considered to be residue one of mature wild-type streptavidin monomer and residue 37 of SEQ ID NO:1 is considered to be residue one of the wild-type core streptavidin sequence. Using this numbering system the residues that are altered in the preparation of some modified monomers of the invention include residues N23, S27, and S45. An example of a modified streptavidin monomer subunit is a D subunit, which includes the following substitutions: N→A at position 23 in the amino acid sequence of mature wild-type streptavidin monomer; S→D at position 27 in the amino acid sequence of mature wild-type streptavidin monomer; and S→A at position 45 in the amino acid sequence of mature wild-type streptavidin monomer.

The sequence set forth as the Dead (D) monomer sequence includes the following substituted amino acid residues: N23A, S27D, and S45A (with the numbering based on the numbering of the mature wild-type streptavidin sequence, which corresponds to amino acids 1-163 of SEQ ID NO:1). A Dead monomer sequence of the invention is set forth herein as SEQ ID NO:3. The sequence of the Alive (A) monomer subunit, which as described above has the unmodified core wild-type streptavidin monomer sequence and may also include a His₆ purification tag. In some embodiments, an Alive (A) monomer does not have a purification tag. The Alive streptavidin monomer subunit with a His₆ tag is set forth herein as SEQ ID NO:4. For use in some methods and preparations of the invention, the sequences set forth as SEQ ID NO:2, 3, and 4 are encoded in a plasmid with an initiating methionine, which is then removed by the E. coli. It will be understood that the presence of an initiating methionine is not an alteration of the core sequence thus is not a modification.

The invention includes in some aspects, a monovalent streptavidin tetramer. A monovalent streptavidin tetramer includes one wild-type streptavidin monomer subunit that maintains wild-type streptavidin binding affinity for biotin or fragments thereof, and three modified streptavidin subunits that have amino acid sequences that are modified from the wild-type streptavidin amino acid sequence. One type of modified streptavidin subunit used in a monovalent streptavidin tetramer of the invention is referred to herein as a Dead (D) type streptavidin monomer subunit and is a modified streptavidin monomer subunit. A preferred monovalent streptavidin tetramer of the invention includes wild-type and modified streptavidin subunits in a 1:3 ratio. A monovalent streptavidin tetramer of the invention contains only a single functional biotin binding subunit. In some preferred monovalent streptavidin tetramers, all three modified streptavidin monomer subunits have the same type of sequence modification. The wild-type streptavidin monomer subunit may also include a purification tag that may be used for the preparation and purification of monovalent streptavidin tetramers.

The wild-type streptavidin monomer subunit may, but need not, include a purification tag that may be used for the preparation and purification of monovalent streptavidin polymers. An example of a methods for purification of a monovalent streptavidin polymer that utilizes a purification tag is provided in the Examples section. An example of one alternative method of purifying a monovalent streptavidin polymer without use of a purification tag includes separation of various tetramers with an iminobiotin column. With an iminobiotin column, D4 tetramers would not bind the column and other streptavidin tetramers would be eluted in the order A1D3, A2D2, A3D1, and then A4 with the later tetramers eluting with decreasing pH. Those of ordinary skill in the art will recognize that additional methods can also be used for separating and/or purifying streptavidin tetramers that have differing ratios of A to D streptavidin subunits.

Functional features of streptavidin polymers and monomers can be determined and are useful for characterizing polymers that include different combinations of wild-type and modified streptavidin monomer subunits. One such functional feature is a the binding affinity for biotin or a fragment thereof of a streptavidin monomer or polymer. As is recognized in the art, binding affinity can be expressed in terms of the dissociation of bound biotin or a fragment thereof from the streptavidin monomer or polymer to which it is bound. Thus, binding affinity can be expressed as the dissociation constant (K_{d}) of binding of a streptavidin monomer or polymer for biotin or a fragment thereof. The binding affinity of a streptavidin monomer or polymer can be determined as described below herein, or using other art-known methods. The binding affinity of a streptavidin monomer or polymer of the invention can be compared to the binding affinity of a wild-type monomer or polymer determined under substantially identical conditions. For example, if wild-type streptavidin tetramer is determined to have a K_{d} of about 4.0×10⁻¹⁴ M when binding biotin or a fragment thereof, a streptavidin tetramer of the invention can be tested under the same conditions to allow a comparison of the affinity of the streptavidin tetramer of the invention to that of a wild-type streptavidin tetramer.

In some instances, it may be desirable to have streptavidin monomers that have reduced level of biotin binding affinity (as compared to wild-type) and to use such a modified streptavidin monomer as a subunit of a streptavidin tetramer. Modified streptavidin monomers are provided herein that have a binding affinity significantly lower than wild-type streptavidin monomer biotin binding affinity. A Dead (D) monomer subunit of the invention has much reduced or no functional binding to biotin or a fragment thereof. A Dead (D) streptavidin monomer subunit may have a K_{d} of about 1 mM. In some embodiments, the K_{d} of a Dead (D) streptavidin subunit is greater than or equal to 5×10⁻⁴ M. In certain embodiments, the K_{d} of a Dead (D) streptavidin subunit is greater than or equal to 5×10⁻³ M (1 mM). In certain embodiments, the K_{d} of a Dead (D) streptavidin subunit is greater than or equal to 1×10⁻³ M. In certain embodiments, the K_{d} of a Dead (D) streptavidin subunit is greater than or equal to 5×10⁻⁴ M. In some embodiments of the invention, the affinity of the Dead (D) streptavidin subunit is so low as to result in essentially no functional binding of the Dead (D) monomer subunit to biotin or a fragment thereof. Thus, a streptavidin tetramer of the invention that includes three Dead (D) streptavidin monomer subunits and one wild-type streptavidin subunit will bind biotin or a fragment thereof at the single wild-type biotin binding site, and thus is defined as a monovalent streptavidin tetramer.

A monovalent streptavidin tetramer of the invention has a single biotin binding site and that has femtomolar binding affinity for biotin or a fragment thereof. As used herein, a femtomolar binding affinity means a K_{d} of from about 1×10⁻¹⁵ M to about 9.99×10⁻¹³ M for that monomer binding site. The Alive (A) subunit in a monovalent streptavidin tetramer of the invention may bind biotin or a fragment thereof with a wild-type streptavidin monomer subunit binding affinity.

A monovalent streptavidin tetramer of the invention may have a proximal streptavidin K_{d} for binding biotin or a fragment thereof. As used herein, the term “proximal streptavidin K_{d}” means having a K_{d} that is between 1×10⁻¹² M and the K_{d} of a wild-type streptavidin tetramer or up to 10-fold lower than the K_{d} for wild-type streptavidin tetramers. Thus, a proximal streptavidin K_{d} may be a level from 1×10⁻¹² down through the K_{d} of wild-type streptavidin tetramer or below wild-type K_{d} to as low as about 4×10⁻¹⁵ M. Thus, a proximal streptavidin K_{d} may be 1×10⁻¹² M, 5×10⁻¹³M, 1×10⁻¹³M, 5×10⁻¹⁴ M or any level in between about 1×10⁻¹²M and about 4×10⁻¹⁵M.

A second functional feature of streptavidin tetramer and monomer binding that can be determined and may be useful to assess various polymers and monomers of the invention is the off-rate of biotin from streptavidin after binding. Off-rate is a measure of time it takes for biotin to dissociate from a streptavidin monomer or polymer to which it has bound. A faster off-rate indicates less stable binding than a streptavidin monomer or polymer with a slower off-rate, which has more stable binding between the biotin and the streptavidin monomer or polymer, respectively. A determination of the off-rate of biotin binding to a streptavidin monomer or polymer thus can provide information regarding the stability of the binding. Off-rate determinations can be made using methods provided in the Examples section as well as using as additional art-known methods of measuring binding dissociation. A streptavidin monomer or tetramer of the invention may have a proximal streptavidin overall biotin off rate. As used herein, the term “proximal streptavidin overall biotin off rate” means that the percentage of biotin dissociation from the streptavidin monomer or tetramer is no more than 1%, 5%, 10%, 15%, 20%, or 25% higher (including all intervening percentages) than the percentage of biotin dissociation from a wild-type streptavidin monomer or tetramer, respectively, under substantially identical conditions. For example, the biotin off rate of a streptavidin tetramer of the invention and a wild-type streptavidin tetramer can be determined under the essentially the same conditions and the percent dissociation of biotin from the streptavidin tetramer will be no more than 1%, 5%, 10%, 15%, 20%, or 25% higher (including all intervening percentages) than the percentage of biotin dissociation from a wild-type streptavidin tetramer.

There are also functional features of streptavidin polymers that may be useful to assess various polymers of the invention. One functional aspect that is useful to assess streptavidin polymers is the stability of streptavidin polymers over time. The stability of streptavidin tetramers involves a determination of whether a polymer would rearrange its subunits over time. A rearrangement of subunits may include a change in the ratio of different types of monomers in a polymer. For example, in a plurality of streptavidin tetramers that have a 3:1 ratio of a modified streptavidin monomers to wild-type streptavidin monomers, the ratio of subunit types may change over time, thus resulting in a mixed population of 3:1, 2:2, and 1:3 ratios of modified to wild-type streptavidin subunits. Low levels of stability result in faster shifts in streptavidin monomer ratios in a streptavidin polymer and higher levels of stability result in reduced changes in the ratio of streptavidin subunit types in a streptavidin polymer.

The thermostability of streptavidin polymers is another functional feature that may be used to assess streptavidin polymers of the invention. Thermal stability can be assessed by heating streptavidin polymers as a determination of whether the streptavidin monomers remain associated in the polymers or dissociate. In some embodiments, dissociation of monomeric subunits of polymers means dissociation into monomeric subunits. In other embodiments, it may mean loss of one or more monomer subunits with dimer or trimer polymers remaining. Methods for determining thermostability of a streptavidin polymer are provided herein and also include additional assessment methods known in the art.

The modified and wild-type monomers and the monovalent and polyvalent streptavidin polymers of the invention may include a tag or label. In some embodiments, a tag is a purification tag. Purification tags of the invention include, but are not limited to polyhistidine tags (e.g. a His₆ tag). Additional types of purification tag sequences are known in the art and may be used in conjunction with the streptavidin tetramers of the invention. Examples of purification tags, although not intended to be limiting, include the HQ tag from Promega (Madison, Wis.) that has a sequence of HQHQHQ, a FLAG tag (DYKDDDDK), or numerous other epitope tags known in the art. See, for example, Jarvik, J. W. and Telmer, C. A. Annu. Rev. Genet. 32: 601-618, 1998.

In some embodiments of the invention, a streptavidin monomer subunit or streptavidin polymer of the invention is linked to a detectable label. Detectable labels useful in the invention include, but are not limited to: a fluorescent label, an enzyme label, a radioactive label, visual label (e.g. a metallic label such as ferritin or gold), a nuclear magnetic resonance active label, an electron spin resonance label, a positron emission tomography label, a luminescent label, and a chromophore label. The detectable labels of the invention can be attached to the streptavidin monomer subunits or streptavidin polymers of the invention by standard protocols known in the art. In some embodiments, the detectable labels may be covalently attached to a streptavidin monomer or polymer of the invention. The covalent binding can be achieved either by direct condensation of existing side chains or by the incorporation of external bridging molecules. In some embodiments a detectable label may be attached to a streptavidin monomer or polymer of the invention using genetic methods. In some embodiments, a label may be attached by conjugating a moiety of interest (e.g. the labeling moiety) to biotin or a biotin analog and then non-covalent binding to the streptavidin polymer. In some embodiments of the invention, more than one type of detectable label may be attached to a streptavidin monomer or polymer of the invention.

The streptavidin monomers and polymers of the invention bind to biotin or fragments thereof. By biotin fragments is meant a fragment of biotin that is sufficiently unchanged from the structure of biotin to be recognized and bound by a streptavidin monomer or polymer of the invention and or by a wild-type monomer or polymer. The biotin fragments may be considered to be functional biotin fragments. By “functional” biotin fragments, is meant that the biotin fragment is recognized by and can be bound by a streptavidin monomer or polymer of the invention.

The invention includes the use of a streptavidin monomer or polymer of the invention to bind to a biotin molecule or fragment thereof that is conjugated to an additional molecule or compound. Examples of such molecules or compounds, though not intended to be limiting, include proteins, nucleic acids, fatty acids, carbohydrates, small molecules, enzymes, antibodies, drug molecules, chemical compounds, cells, etc. Biotin is extensively used by those of skill in the scientific arts in labeling and tracking methods. Therefore, one of ordinary skill the art will recognize that the streptavidin molecules of the invention may be used to bind numerous different types of biotin conjugates. In some embodiments, a biotin or biotin molecule (conjugated or not) may be in solution or may be attached to a surface. Examples of surfaces to which a streptavidin polymer of the invention may be attached include, but are not limited to, a magnetic or chromatographic bead or particle bead or a chromatography support or other support.

The streptavidin monomers and polymers of the invention can be used for a wide variety of purposes including, but not limited to: cell sorting, cell labeling, drug delivery, imaging methods, etc. The streptavidin polymers of the invention can be linked to labels, delivery molecules, cells, etc for use in various technologies. The streptavidin polymers of the invention are also useful for imaging, including real-time imaging in vitro and in vivo.

An example of the use of a streptavidin polymer of the invention, though not intended to be limiting, is the use of the streptavidin polymer to isolate a target molecule or compound from a complex mixture or solution. In such embodiments, a streptavidin polymer of the invention can be attached to a targeting molecule and contacted with the complex mixture. The targeting molecule, attached to the streptavidin polymer, binds to the target and the complex mixture can be contacted with biotin or a fragment thereof, either alone, or in a conjugated form. Binding of the streptavidin polymer of the invention to the biotin or fragment thereof enables detection of the bound target in the complex mixture. Additionally, standard separation methods can then be used to separate the bound target molecule or compound from the complex mixture using standard separation methods.

Another example of methods in which the streptavidin polymers of the invention, including the monovalent streptavidin tetramers described herein, may be used is in single-particle tracking, which is described in the Examples section herein. Additional labeling, imaging, cell sorting, and delivery methods for which the streptavidin polymers of the invention may be used, include a wide variety of art-known methods that include the use of streptavidin/biotin interactions.

Additional uses for streptavidin monomers and polymers of the invention may also include control of assembly of nanodevices. For example, binding of controlled numbers of biotinylated DNA, biotinylated proteins or biotinylated inorganic particles (including carbon nanotubes and quantum dots) to a surface or bead for systems detecting biological analytes or for building electrical circuits. An example of an application is the targeting to chemically biotinylated erythrocytes of streptavidin bound to drugs or other proteins. Erythrocytes may be used as drug delivery vehicles and may be useful because of their long circulation time. Unlike this method of targeting with the streptavidin polymers of the invention, previous targeting with biotin binding proteins has caused complement lysis of the erythrocytes from cross-linking of surface proteins (Muzykantov, V. R. et al., Anal Biochem 241(1): 109-119, 1996.

The invention will be more fully understood by reference to the following examples. These examples, however, are merely intended to illustrate the embodiments of the invention and are not to be construed to limit the scope of the invention.

### EXAMPLES

Avidin Methods

General

Biotin (a gift from Tanabe USA; San Diego, Calif.) was dissolved in Dimethyl Sulfoxide (DMSO) at 100 mM. Sodium dodecyl sulfate-polyacrylamide gel electrophoresis (SDS-PAGE) was performed at 200 V with the gel box (X Cell SureLock, Invitrogen; Carlsbad, Calif.) surrounded by ice to prevent dissociation of the avidin subunits during electrophoresis.

Plasmid Construction

Mutations in avidin were selected and sequences generated by QuikChange™ (Stratagene; La Jolla, Calif.) using appropriate primers and their reverse complements to introduce N12A, S16D, and T35A. Mutations were confirmed by DNA sequencing. To generate the Alive avidin subunit (A), six histidine residues were added to the C-terminus of wild type core sequence by polymerase chain reaction (PCR) of avidin using suitable primers. This PCR product was cloned into sites on the plasmid.

Avidin Expression and Purification

An overnight culture picked from a freshly grown colony of E. coli BL21 (DE3) is diluted 100-fold into LB ampicillin and grown to OD₆₀₀ 0.9 at 37° C. It is then induced with 0.5 mM isopropyl-beta-D-thiogalactopyranoside (IPTG) and incubated for a further 4 hr at 37° C. Inclusion bodies are purified using B-PER (Pierce; Rockford, Ill.), following manufacturer's instructions, and dissolved in 6 M guanidinium hydrochloride pH 1.5 (GuHCl). To generate chimeric avidins, the relative concentration of each unfolded subunit is estimated from OD₂₈₀ in GuHCl and subunits are mixed in the desired ratio. Subunits in GuHCl are refolded by rapid dilution into PBS, and concentrated by ammonium sulphate precipitation, following Schmidt et al. (Schmidt, T. G. and Skerra, A. J. Chromatogr. A 676: 337-345, 1994). The precipitate is re-dissolved in PBS and dialyzed three times against PBS. This step is sufficient to purify wild-type avidin, A4 and D4. To purify chimeric avidins, a Poly-Prep column (Bio-Rad; Hercules, Calif.) is loaded with 1.6 mL Ni-nitrilotriacetic acid agarose (Qiagen; Valencia, Calif.) and is washed with 8 mL of binding buffer (50 mM Tris base, 300 mM NaCl, pH 7.8), using gravity flow at room temperature. The avidin is diluted two-fold in binding buffer and loaded on the column. The column is washed with 8 mL washing buffer (binding buffer+10 mM imidazole) and then with 8 mL elution buffer 1 (binding buffer+70 mM imidazole, eluting principally A1D3). 0.5 mL fractions are collected from this elution and from subsequent elutions in 8 mL elution buffer 2 (binding buffer+100 mM imidazole, eluting principally A2D2) or 8 mL elution buffer 3 (binding buffer+125 mM imidazole, eluting principally A3D1). Fractions are mixed with 6×SDS-loading buffer (0.23 M Tris HCl pH 6.8, 24% v/v glycerol, 120 μM bromophenol blue, 0.4 M dithiothreitol, 0.23 M SDS) and are loaded without boiling onto 8% SDS-PAGE gels. Fractions of the correct composition, determined by comparison to the bands from the initial refold, are pooled and dialyzed in PBS. Avidin concentration is determined in PBS from OD₂₈₀ using ε₂₈₀ of 34,000M⁻¹ cm⁻¹ (Sano, T. and Cantor, C. R. Proc. Natl. Acad. Sci. USA 87: 142-146, 1990). Where required, samples are concentrated using a Centricon Ultracel YM10 (Millipore; Billerica, Mass.).

Avidin polymers are also purified by use of an iminobiotin column. For example, using this separation method various avidin tetramers with different ratios of D:A avidin subunits are separated from each other without the need to include a purification tag on the avidin tetramer. Dead (D4) tetramers, which have four D avidin subunits. do not bind the column and other avidin tetramers are eluted in the order A1D3, A2D2, A3D1, and then A4 under decreasing pH conditions. Standard elution conditions are used. Iminobiotin is available from Pierce Biotechnology, Inc, Rockford, Ill.

Fluorophore Conjugation to Avidin

Avidin and its variants are labeled with Alexa Fluor 568 by adding 1/10 volume of 1 M NaHCO₃ pH 8.4 and then a 10-fold molar excess of Alexa Fluor 568 succinimidyl ester (Molecular Probes; Carlsbad, Calif.) (stock dissolved at 1 mg/mL in dry dimethylformamide) and incubating for 4 hr at room temperature. Free dye is separated on a NAP5 column (GE Healthcare; Amersham Biosciences, Piscataway, N.J.) following manufacturer's instructions. Fractions containing labeled protein, determined by running boiled samples on a 16% SDS-PAGE gel, are pooled and dye is further removed by two rounds of dialysis in PBS.

Mass Spectrometry

Biospin columns (Bio-Rad) are equilibrated by spinning 5 times in 500 μL of 15 mM ammonium acetate pH 7.8 at 1000 g for 2 min. Then 30-50 μL of 30 μM protein in PBS is buffer-exchanged into 15 mM ammonium acetate pH 7.8 using the pre-equilibrated Biospin columns by spinning for 20 s at 1000 g. To ensure that PBS is completely removed, the flow-through is again buffer-exchanged with a second pre-equilibrated column for 20 s. This procedure also removes free biotin when the starting 30 μM avidin forms are incubated with 200 μM biotin. Less than 2 min before introducing into the mass spectrometer, the buffer-exchanged samples are diluted with a solution of 1:1 15 mM ammonium acetate pH 7.8 and 78% acetonitrile, 0.01% trifluoroacetic acid.

An Advion nanospray robot (Advion BioSystems, Ithica, N.Y.) with a back-pressure of 0.45 Psi introduces the samples into the mass spectrometer, an 8.5 Tesla custom-built Electrospray Ionisation-Fourier Transform Mass Spectrometer. To visualize the non-covalent tetramers and non-covalent biotin binding in the high m/z range, the following settings are used: Chirp rate=750 Hz, Amplitude=0.5 V p-p, Tube lens=200 V, Capillary heater 2 V, Quad filter=−20 V, Skimmer=0 V, Capillary offset=34 V, X-fer=−110 V, Leak gas=4.2×10⁻⁵ Torr. The capillary heater is kept low and the Quad filter and Skimmer are kept either high or off to prevent subunit dissociation. The transfer is set to this low value of −110 V in order to visualize the high m/z region.

The masses are calculated manually by first determining the charge state. The final mass is determined by multiplying the observed m/z by the charge and subtracting the mass corresponding to the addition of protons to give that charge. This calculation is repeated for each charge state and the mean and standard deviation reported. The spectra are calibrated with tetrameric avidin, after its monomer mass is determined under denaturing conditions. Average masses are predicted from the DNA sequence, using the ExPASy PeptideMass Calculator (ca.expasy.org/tools/peptide-mass.html) and assuming removal of the N-terminal formyl-Methionine.

K_{d} Measurements

The K_{d} of A1D3 avidin (A is “Alive” subunit; D is “Dead” subunit) is obtained using a competition assay modified from Klumb et al. (Klumb, L. A. et al. Biochemistry 37: 7657-7663, 1998). Wild-type avidin is depleted of the small amount of co-purifying monomeric avidin by gel filtration. Fully tetrameric wild-type avidin (60 nM each subunit) is mixed with 20 nM (Kada, G. et al., Biochim. Biophys. Acta 1427: 33-43, 1999; Bayer, E. A. et al., Electrophoresis 17: 1319-1324, 1996) ³H-biotin (Amersham; Piscataway, N.J.) and 0-1.4 μM of competing A1D3 in PBS pH 7.0. Mixtures are incubated at 37° C. for >20 hr to allow sufficient time for equilibration. To separate the His₆-tagged A1D3 from wild-type avidin, an equal volume of a 50% slurry of Ni-NTA beads (Qiagen) in PBS with 15 mM imidazole is added. After 1 hr at room temperature, the beads are cleared by centrifugation at 15,600 g for 1 min. Aliquots are taken from the supernatant containing the biotin-bound wild-type avidin, an equal volume of 10% SDS in water is added, and samples are heated to 95° C. for 30 min, and counted in a Beckman Coulter LS6500 Liquid Scintillation Counter. The K_{d} ratio is obtained using Matlab (Mathworks; Natick, Mass.) using the formula from Klumb et al. (Klumb, L. A. et al. Biochemistry 37: 7657-7663, 1998). The affinity of A1D3 is calculated from this K_{d} ratio multiplied by the previously determined K_{d} of wild-type avidin for biotin of 4×10⁻¹⁴M and divided by four, since only one of the four subunits of A1D3 showed significant biotin binding.

It is difficult to detect biotin binding by D4 using a competition assay against wild-type avidin because of its extremely low binding affinity, and so the following assay is used instead to determine the K_{d} (Reznik, G. O. et al. Proc. Natl. Acad. Sci. USA 95: 13525-13530, 1998): 24 μM D4 is incubated with 0-500 μM ³H-biotin in 100 μL total volume. After incubation at room temperature for 20 hr, the protein is precipitated by adding 50 μL to 200 μL 0.2 M ZnSO₄ followed by 200 μL 0.2 M NaOH. The protein precipitate is pelleted by centrifugation at 16,500 g for 5 min. The biotin bound by D4 is calculated from the total ³H-biotin added minus the ³H-biotin in the supernatant. The K_{d} is obtained using a nonlinear regression analysis (one-site binding hyperbola) with SigmaPlot (Systat Software; Point Richmond, Calif.).

Off-Rate Assay

The off-rate of biotin-fluorescein from avidin is measured in PBS with 20 mM HEPES pH 7.4 (PBS-H) using a Safire plate-reader and XFluor4 software (Tecan US; Durham, N.C.) with 494 nm excitation and 527 nm emission. In this assay the binding of biotin-4-fluorescein to an excess of avidin results in quenching of fluorescein emission (Kada, G. et al. Biochim. Biophys. Acta 1427: 33-43, 1999). As the biotin-4-fluorescein dissociates, the fluorescence recovers. The assay is performed in the presence of excess biotin so that sites left open by biotin-4-fluorescein dissociation are immediately re-filled by biotin. Avidin tetramer at 1 μM in 10 μL PBS-H is added to 12 nM biotin-4-fluorescein (Molecular Probes) in 170 μL PBS-H and incubated for 30 min at 37° C. 20 μL PBS-H or 20 μL PBS-H 10 mM biotin is then added and recording immediately started, with incubation at 37° C. Percentage dissociation is calculated as (signal with biotin−signal without biotin)/(mean maximal signal of T90I with biotin−initial T90I signal without biotin)×100. The concentration of competing biotin is saturating, since reducing the biotin concentration ten-fold produced indistinguishable dissociation rates.

Thermostability Assay

2.3 μM wild-type avidin or chimeric avidin in PBS is heated at the indicated temperature for 3 min in a PTC-200 PCR machine (MJ Research; Waltham, Mass.) and then immediately placed on ice (Bayer, E. A. et al. Electrophoresis 17: 1319-1324, 1996). Samples are mixed with 6×SDS-PAGE loading buffer and loaded onto a 16% polyacrylamide gel.

Cell Culture, Biotinylation and Imaging

HeLa cells are grown in Dulbecco's Modified Eagle Medium (DMEM) with 10% Fetal Calf Serum, 50 U/mL penicillin and 50 μg/mL streptomycin. HeLa stably expressing AP-CFP-TM or Ala-CFP-TM have been previously described (Howarth, M. et al., Proc. Natl. Acad. Sci. USA 102: 7583-7588, 2005). Dissociated primary neuronal cultures are prepared from Embryonic Day 18 or 19 (E18/19) rats and transfected with Lipofectamine 2000 at DIV6 as in Levinson et al. (Levinson, J. N. et al. J. Biol. Chem. 280: 17312-17319, 2005).

Enzymatic biotinylation and imaging of HeLa transfectants are performed as previously described (Howarth, M. et al., Proc. Natl. Acad. Sci. USA 102: 7583-7588, 2005), except instead of 10 μM biotin and 1 mM ATP, we add 10 μM biotin-AMP (synthesized according to Coleman and Huang; Coleman, T. M. and Huang, F. Chem. Biol. 9: 1227-1236, 2002) to give equivalent biotinylation but minimizing the risk of purinoreceptor activation by ATP (Rathbone, M. P. et al., Prog. Neurobiol. 59: 663-690, 1999). HeLa transfectants are biotinylated for 10 min at room temperature, and stained with 10 μg/mL Alexa Fluor 568-conjugated wild-type avidin, D4 or A1D3 for 10 min at 4° C. Biotinylation of neurons is performed at day in vitro (DIV) 8 in Hanks' Balanced Salt Solution (HBSS) (Invitrogen) with 0.2 μM biotin ligase and 10 μM biotin-AMP for 5 min at 37° C. Neurons are then washed with HBSS and incubated for 2 min with 5 μg/mL Alexa Fluor 568-conjugated wild-type avidin (Molecular Probes) or A1D3 at 37° C. Neurons are washed with NeuroBasal media (Invitrogen) supplemented with B-27 (Invitrogen), 50 U/mL penicillin, 50 μg/mL streptomycin, and 0.2 mM L-glutamine and chased in the same medium for 0 or 2 hr at 37° C. Cells are then fixed in −20° C. methanol. There was no signal from wild-type avidin labeling if neurons were instead transfected with Ala-neuroligin-1 containing a point mutation in AP, confirming the specificity of labeling. To observe synapse formation, cells are biotinylated and stained with avidin as above, biotinylation and avidin staining is repeated at 6 hr, and then after 24 hr total chase cells are fixed in methanol. Samples are stained for pre-synaptic markers using guinea pig anti-VGLUT1 (1:1000, Chemicon; Temecula, Calif.), followed by goat anti-guinea pig Alexa Fluor 488 (1:1000, Molecular Probes). All antibody reactions are performed in blocking solution [PBS with 0.3% Triton X-100 and 2% normal goat serum (Vector Laboratories; Burlingame, Calif.)] for 1 hr at room temperature or overnight at 4° C.

Images of HeLa cells are collected on a Zeiss Axiovert 200M inverted epifluorescence microscope using a 40× oil-immersion lens and a MicroMAX CCD camera (Roper Scientific; Tucson, Ariz.). CFP (420DF20 excitation, 450DRLP dichroic, 475DF40 emission) and Alexa568 (560DF20 excitation, 585DRLP dichroic, 605DF30 emission) images are collected and analyzed using OpenLab software (Improvision; Lexington, Mass.). Fluorescence images are background-corrected. Neuron are were acquired on a Zeiss Axiovert 200M microscope with a 63×1.4 NA Acromat oil-immersion lens and a monochrome 14-bit Zeiss Axiocam HR charged-coupled camera with 1300×1030 pixels. To correct for out-of-focus clusters within the field of view, focal plane z-stacks are acquired and maximum intensity projections performed off-line. Images are scaled to 16 bits and analyzed in Northern Eclipse (Empix Imaging; Ontario, Canada) with user-written software. Briefly, images are processed at a constant threshold level (of 32,000 pixel values) to create a binary mask image, which is multiplied with the original image using Boolean image arithmetic. The resulting image contained a discrete number of clusters with pixel values of the original image. Only dendritic clusters greater than 5 pixels in size, and with an average pixel values 2 times greater than background pixel values are used for analysis. Results are then calculated in terms of clusters per micrometer of dendrite. For assessment of pre-synaptic terminals, clusters are determined as before and average grey levels of clusters are compared between transfected dendrites and untransfected dendrites within the same field of view. The two-tailed parametric Student's t-test is performed to calculate statistical significance of results between experimental groups. “n” represents the number of transfected neurons for which clusters were measured.

### Example 1

Methods provided in the Methods section above are used to make and test the monovalent avidin. We produce an avidin tetramer consisting of three subunits unable to bind biotin and one subunit that binds biotin as well as wild-type avidin. A triple mutant N12A, S16D, T35A is produced. The triple mutant N12A, S16D, T35A shows negligible biotin binding and leaves the tetramer structure intact. The binding of this triple mutant (composed of “Dead” subunits-D) is so weak that it is difficult to measure. To generate monovalent avidin the wild-type subunit is first tagged with a His₆-tag (“Alive” subunit-A). Then D and A subunits are combined at a molar ratio of 3:1 in guanidinium hydrochloride and refolded by rapidly diluting the mixture into PBS. This refold generates a mix of avidin tetramers of different compositions.

The different tetramers are purified using a Ni-nitrilotriacetic acid (NTA) column, eluting according to the number of His₆-tags with increasing concentrations of imidazole. The tetramers can also be distinguished by SDS-PAGE, if the samples are not boiled, according to the number of His-tags present, showing that at least 30% are of the monovalent A1D3 form. Thus purified fractions of the monovalent A1D3 are obtained, as well as the other chimeric avidins, A2D2 and A3D1. The tetramer composition is further confirmed by boiling the samples before loading on SDS-PAGE, to determine the ratio of A to D subunits, and by electrospray ionization mass spectrometry. The observed mass (±s.d.), determined by Electrospray Ionization-Mass Spectrometry, is compared to the mass predicted from the sequence. From the change (±s.e.m.) upon addition of biotin, we determine how many biotin molecules are bound to each tetramer.

Despite the large mass of the avidin tetramer and non-covalent interaction between subunits, good agreement is found between expected and observed masses for D4, A1D3, A2D2, A3D1 and A4.

### Example 2

Methods described in the Avidin Methods section above herein are used for the following production and testing of monovalent avidin. Tests are performed to determine whether monovalent avidin will rearrange its subunit composition over time. A1D3 is incubated at room temperature or at 37° C. and analyzed by SDS-PAGE, to look for the appearance of D4 and A2D2 from subunit exchange. A small percentage of the A1D3 rearranges into D4 after 37° C. incubation for one day or after room temperature incubation for one week. Formation of A2D2 is not detected in either case, indicating that significant fractions of multivalent avidin are not generated upon storage. Next the stability of A1D3 to dissociate into monomers is tested, since many mutations in the biotin binding site of avidin weaken tetramer stability. Wild-type avidin and A1D3 are heated in PBS at various temperatures and tetramer disassembly is determined by SDS-PAGE. A significant fraction of A1D3 remains tetrameric even at 100° C. There is little difference in thermostability between wild-type avidin and monovalent avidin, suggesting that the mutations in D have minimal effect on the subunit interfaces and that it should be possible to use A1D3 in assays requiring high temperatures.

Electrospray ionization mass spectrometry is used to characterize the number of biotin molecules bound per avidin tetramer. Spectra of the different avidin tetramers with or without biotin are acquired. As expected, all four subunits of A4 are associated with biotin. No biotin binding by D4 is detected. A1D3 is monovalent, binding a single biotin. The other chimeric tetramers bind one biotin per A subunit.

The biotin binding affinity of A1D3 is determined by measuring competition with wild-type avidin for ³H biotin. The stability of biotin-conjugate binding to A1D3 is also evaluated. An avidin mutant found to have a fast off-rate is used as a positive control for biotin-conjugate dissociation.

To determine the off-rate of biotin from A1D3, 10 nM ³H-biotin is pre-incubated with 1 μM A1D3 or wild-type avidin for 20 minutes at 37° C. Dissociation is then initiated by addition of cold biotin at a final concentration of 50 μM and time-points are taken over 5 hours at 37° C. 50 μL aliquots are removed and added to 200 μL 0.2M ZnSO₄ chilled on ice, followed by 200 μL 0.2 M NaOH. The protein precipitate is pelleted by centrifugation at 16,500 g for 5 min, and ³H-biotin in the supernatant is measured by liquid scintillation counting. Data are plotted as ln(fraction bound) versus time, and fit to a straight line by linear regression. Dissociation rates are deduced from the slope of the line and the equation:
ln(fraction bound)=−k_{off}(t)
where fraction bound=(total ³H-biotin−free ³H-biotin at timepoint)/(total ³H-biotin−free ³H-biotin before cold biotin chase).

Site-specific biotinylation is used to study cell surface protein trafficking (Howarth, M. et al., Proc. Natl. Acad. Sci. USA 102: 7583-7588, 2005). Proteins of interest are tagged with a 15 amino acid acceptor peptide (AP), which is biotinylated by incubating cells with biotin ligase (BirA). The biotinylated protein is then tracked by labeling with fluorophore- or quantum dot-conjugated avidin (Howarth, M. et al., Proc. Natl. Acad. Sci. USA 102: 7583-7588, 2005), resulting in an interaction that is stable for many hours, unlike antibody labeling or other non-covalent site-specific labeling methods (Chen, I. and Ting, A. Y. Curr. Opin. Biotechnol. 16: 35-40, 2005).

Labeling of site-specifically biotinylated cell surface proteins with monovalent avidin is performed. Cyan fluorescent protein is tagged with AP and targeted to the surface of HeLa cells with a transmembrane domain (AP-CFP-TM). CFP-TM is cyan fluorescent protein with an acceptor peptide (AP), targeted to the cell surface with the transmembrane helix of PDGF receptor. HeLa expressing AP-CFP-TM or Ala-CFP-TM (a control with an alanine point mutation in AP) are biotinylated with biotin ligase for 10 min and stained with wild-type or monovalent (A1D3) avidin conjugated to Alexa Fluor 568. The Alexa-labeled and CFP-labeled images are overlaid. The results indicate no Alexa staining of AP-CFP-TM is observed when D4 was used or when biotin ligase is omitted and the cells are labeled with A1D3. Thus, after brief incubation with biotin ligase, biotinylated AP-CFP-TM is detected equally well with wild-type avidin or A1D3. However, an equivalent dye-conjugate of D4 gives no detectable staining, indicating that binding of A1D3 is only through the A subunit. A point mutation in the acceptor peptide (Ala-CFP-TM) that blocks biotin ligase recognition abolishes all staining. Staining is also abolished by omission of biotin ligase. Thus monovalent avidin does not give increased background in cell staining experiments compared to wild-type avidin.

### Example 3

Cross-linking is a central method of control of signal transduction, for example in the activation of growth factor receptors and transcription factors (Klemm, J. D. et al., Annu. Rev. Immunol. 16: 569-592, 1998), but is a concern when labeling cells with antibodies. Although Fab antibody fragments could be used to avoid cross-linking, Fabs are rarely of high affinity (making it difficult to label low abundance antigens) and will tend to dissociate on the time-scale of minutes. Cross-linking is disastrous for single-particle tracking experiments because the presence of an extra anchor slows protein diffusion (Iino, R. et al., Biophys J. 80: 2667-2677, 2001). It is normally said that using a ligand in excess will minimize cross-linking. However, labeled ligand must be present at a density of <1 per μm² for individual particles to be resolved. Thus these two requirements are only compatible if one is studying a target protein present at very low levels. There is still a need for a way to label surface proteins with an interaction of high stability that does not cross-link.

Methods described in the Avidin Methods section above are used for the following neuroligin tests. Neuroligins are post-synaptic adhesion proteins that play a role in the development of excitatory and inhibitory synapses (Scheiffele, P. et al., Cell 101: 657-669, 2000; Levinson, J. N. et al., J. Biol. Chem. 280: 17312-17319, 2005). Clustering of neuroligin has been observed during synapse development, but neuroligin's role in synapse initiation versus synapse stabilization is not clear.

To examine the effect of artificially-induced neuroligin clustering, AP-neuroligin-1 is site-specifically biotinylated at the cell surface with biotin ligase, and detected with either wild-type or monovalent avidin. Hippocampal neurons are transfected with AP-neuroligin-1, biotinylated with biotin ligase, and labeled with Alexa Fluor 568-conjugated wild-type avidin or A1D3. Cells are incubated for 0 or 2 hr at 37° C. and Alexa staining is visualized by fluorescence microscopy. Neurons are biotinylated and labeled with wild-type avidin or A1D3 as above, but incubated for 24 hr and then stained for the pre-synaptic marker VGLUT1. Avidin and VGLUT1 signals are assessed and their images overlaid for comparison. It is determined that the AP-neuroligin-1 clusters are not apposed to pre-synaptic terminals. At zero hours, diffuse surface staining of AP-neuroligin-1 is observed with both wild-type and monovalent avidin. After a two hour incubation, however, monovalent avidin-labeled AP-neuroligin-1 is still predominantly diffuse, but wild-type avidin-labeled AP-neuroligin-1 have formed distinct aggregates, consistent with tetramer-induced protein cross-linking. The same staining pattern is observed after 24 hour incubation.

The aggregation of AP-neuroligin-1 by wild-type avidin correlates with reduced formation of excitatory pre-synaptic contacts, determined by the intensity of vesicular glutamate transporter-1 clusters (VGLUT1), and by the fact that many of the aggregates induced by wild-type avidin are not apposed by pre-synaptic terminals positive for VGLUT1. Thus induction of neuroligin clustering by wild-type avidin had a deleterious effect on pre-synaptic differentiation. The increase in VGLUT1 cluster intensity for neurons transfected with AP-neuroligin-1 and labeled with monovalent avidin is similar to the increase seen for HA-neuroligin-1 transfected neurons (Prange, O. et al., Proc. Natl. Acad. Sci. USA 101: 13915-13920, 2004), suggesting that AP, biotin, and monovalent avidin do not disrupt the function of neuroligin-1 or neuroligin-neurexin interactions. Taken together with previous observations, these results suggest that while gradual neuroligin clustering from DIV7 to DIV14 may promote pre-synaptic differentiation, rapid clustering does not. These results also indicate that monovalent avidin can efficiently label proteins on the neuron surface, while avoiding the complications of aggregation of its target.

### Example 4

The need to purify different chimeric forms of avidin can be avoided if the four subunits can be genetically joined to make a single-chain avidin. However, the distance between the termini means that long linkers would be required, which are likely to impair folding. Attempts to circumvent this problem by circularly permuting avidin have yielded forms with K_{d}>10⁻⁸M (Chu, V. et al., Protein Sci. 7: 848-859, 1998; Aslan, F. M. et al., Proc. Natl. Acad. Sci. USA 102: 8507-8512, 2005). A circularly permuted tetravalent single-chain avidin with wild-type binding affinity was very recently generated (Nordlund, H. R. et al., Biochem., 2005). However, the ability to inactivate individual binding sites in this single-chain avidin has not been previously demonstrated.

Tetravalent single chain avidin has been produced by Nordlund, et al., Biochemical Journal, published online, Aug. 10, 2005. Circularly permuted avidin is produced by joining the nucleotide sequences of avidin monomer subunits with linkers for expression as a single polypeptide chain. Wild-type avidin monomer subunit sequences are used to produce a single-chain produced wild-type avidin tetramer. The wild-type single chain-produced avidin has four wild-type binding domains.

A monovalent single chain-produced avidin tetramer is also produced. To make the monovalent single chain-produced avidin tetramer, the nucleotide sequence that encodes one wild-type avidin monomer subunit and the three copies of the nucleotide sequence that encodes a modified avidin monomer subunit are linked and a circularly permuted avidin is produced. The monovalent single chain-produced avidin tetramer has a binding affinity of wild-type avidin tetramer. The modified avidin subunits each have a sequence that includes at least three substituted amino acids. The substituted amino acids are N12A, S16D, and T35A. The modified avidin monomers do not functionally bind biotin when part of the monovalent single chain-produced avidin tetramer.

To make avidin dimer or trimer molecules, the nucleotide sequences of two or three avidin monomer subunits (respectively) are joined by linkers and a circularly permuted avidin dimer or trimer molecule is produced. The avidin monomer subunits are either wild-type or modified avidin monomer subunits. A modified avidin subunit includes the three substituted amino acids N12A, S16D, and T35A.

Streptavidin Methods

General

Biotin (a gift from Tanabe USA; San Diego, Calif.) was dissolved in Dimethyl Sulfoxide (DMSO) at 100 mM. Sodium dodecyl sulfate-polyacrylamide gel electrophoresis (SDS-PAGE) was performed at 200 V with the gel box (X Cell SureLock, Invitrogen; Carlsbad, Calif.) surrounded by ice to prevent dissociation of the streptavidin subunits during electrophoresis.

Plasmid Construction

Wild-type core streptavidin in pET21a(+) (Novagen; San Diego, Calif.) was a kind gift from P. Stayton (University of Washington; Seattle, Wash.) (Klumb, L. A. et al., Biochemistry 37: 7657-7663, 1998). Mutations in streptavidin were generated by QuikChange™ (Stratagene; La Jolla, Calif.) using the following primers and their reverse complements: 5′-GGCACCTGGTACGCCCAGCTGGGAGACACCTTCATCGTTAC-3′ (SEQ ID NO:6) to introduce N23A and S27D, 5′-TCTGACCGGTACCTACGAAGCCGCTGTTGGTAACGCTGAAT-3′ (SEQ ID NO:8) to introduce S45A, and 5′ CGCTCACTCCGCTATCACCTGGTCTGGCC-3′ (SEQ ID NO:10) to introduce T901. Mutations were confirmed by DNA sequencing. The reverse complements of SEQ ID NO:6, 8, and 10 are SEQ ID NO:7, 9, and 11, respectively. To generate the Alive streptavidin subunit (A), six histidine residues were added to the C-terminus by polymerase chain reaction (PCR) of streptavidin using the primers 5′-TCCAGAATTCGTAACTAACTAAAGGAGA (SEQ ID NO:12) and 5′-AGACAAGCTTTTATTAATGGTGGTGATGGTGATGGGAAGCAGCGGACGGTTT-3′ (SEQ ID NO:13). This PCR product was cloned into the BamHI and HindIII sites of pET21a(+)-streptavidin (Klumb, L. A. et al., Biochemistry 37: 7657-7663, 1998). AP-neuroligin was generated from pEGFP-G1 containing mouse neuroligin-1 (Levinson, J. N. et al., J. Biol. Chem. 280: 17312-17319, 2005) by replacing Green Fluorescent Protein (GFP) with the acceptor peptide (AP) (Chen, I. et al., Nat. Methods 2: 99-104, 2005) at the AgeI and BglII sites, using the primers: 5′-CCGGTCGGCCTGAACGATATCTTCGAGGCCCAGAAGATCGAGTGGCACGAGA-3′ (SEQ ID NO:14) and 5′-GATCTCTCGTGCCACTCGATCTTCTGGGCCTCGAAGATATCGTTCAGGCCGA-3′, (SEQ ID NO:15) so that AP would be at the N-terminus of neuroligin-1. To make Ala-neuroligin, a lysine in the AP was mutated to alanine by QuikChange™ using the primers described in Chen et al. (Chen, I. et al., Nat. Methods 2: 99-104, 2005). The construction of AP-CFP-TM and Ala-CFP-TM plasmids has been described (Chen, I. et al., Nat. Methods 2: 99-104, 2005).

Streptavidin Expression and Purification

An overnight culture picked from a freshly grown colony of E. coli BL21(DE3) was diluted 100-fold into LB ampicillin and grown to OD₆₀₀ 0.9 at 37° C. It was then induced with 0.5 mM isopropyl-beta-D-thiogalactopyranoside (IPTG) and incubated for a further 4 hr at 37° C. Inclusion bodies were purified using B-PER (Pierce; Rockford, Ill.), following manufacturer's instructions, and dissolved in 6 M guanidinium hydrochloride pH 1.5 (GuHCl). To generate chimeric streptavidins, the relative concentration of each unfolded subunit was estimated from OD₂₈₀ in GuHCl and subunits were mixed in the desired ratio. Subunits in GuHCl were refolded by rapid dilution into PBS, and concentrated by ammonium sulphate precipitation, following Schmidt et al. (Schmidt, T. G. and Skerra, A. J. Chromatogr. A 676: 337-345, 1994). The precipitate was re-dissolved in PBS and dialyzed three times against PBS. This step was sufficient to purify wild-type streptavidin, A4 and D4. To purify chimeric streptavidins, a Poly-Prep column (Bio-Rad; Hercules, Calif.) was loaded with 1.6 mL Ni-nitrilotriacetic acid agarose (Qiagen; Valencia, Calif.) and was washed with 8 mL of binding buffer (50 mM Tris base, 300 mM NaCl, pH 7.8), using gravity flow at room temperature. The streptavidin was diluted two-fold in binding buffer and loaded on the column. The column was washed with 8 mL washing buffer (binding buffer+10 mM imidazole) and then with 8 mL elution buffer 1 (binding buffer+70 mM imidazole, eluting principally A1D3). 0.5 mL fractions were collected from this elution and from subsequent elutions in 8 mL elution buffer 2 (binding buffer+100 mM imidazole, eluting principally A2D2) or 8 mL elution buffer 3 (binding buffer+125 mM imidazole, eluting principally A3D1). Fractions were mixed with 6×SDS-loading buffer (0.23 M Tris HCl pH 6.8, 24% v/v glycerol, 120 μM bromophenol blue, 0.4 M dithiothreitol, 0.23 M SDS) and were loaded without boiling onto 8% SDS-PAGE gels. Fractions of the correct composition, determined by comparison to the bands from the initial refold, were pooled and dialyzed in PBS. Streptavidin concentration was determined in PBS from OD₂₈₀ using ε₂₈₀ of 34,000M⁻¹ cm⁻¹ (Sano, T. and Cantor, C. R. Proc. Natl. Acad. Sci. USA 87: 142-146, 1990). Where required, samples were concentrated using a Centricon Ultracel YM10 (Millipore; Billerica, Mass.).

Streptavidin polymers are also purified by use of an iminobiotin column. For example, using this separation method various streptavidin tetramers with different ratios of D:A avidin subunits are separated from each other without the need to include a purification tag on the streptavidin tetramer. Dead (D4) tetramers, which have four D streptavidin subunits. do not bind the column and other streptavidin tetramers are eluted in the order A1D3, A2D2, A3D1, and then A4 under decreasing pH conditions. Standard elution conditions are used. Iminobiotin is available from Pierce Biotechnology, Inc, Rockford, Ill.

Fluorophore Conjugation to Streptavidin

Streptavidin and its variants were labeled with Alexa Fluor 568 by adding 1/10 volume of 1 M NaHCO₃ pH 8.4 and then a 10-fold molar excess of Alexa Fluor 568 succinimidyl ester (Molecular Probes; Carlsbad, Calif.) (stock dissolved at 1 mg/mL in dry dimethylformamide) and incubating for 4 hr at room temperature. Free dye was separated on a NAP5 column (GE Healthcare; Amersham Biosciences, Piscataway, N.J.) following manufacturer's instructions. Fractions containing labeled protein, determined by running boiled samples on a 16% SDS-PAGE gel, were pooled and dye was further removed by two rounds of dialysis in PBS.

Mass Spectrometry

Biospin columns (Bio-Rad) were equilibrated by spinning 5 times in 500 μL of 15 mM ammonium acetate pH 7.8 at 1000 g for 2 min. Then 30-50 μL of 30 μM protein in PBS was buffer-exchanged into 15 mM ammonium acetate pH 7.8 using the pre-equilibrated Biospin columns by spinning for 20 s at 1000 g. To ensure that PBS was completely removed, the flow-through was again buffer-exchanged with a second pre-equilibrated column for 20 s. This procedure also removed free biotin when the starting 30 μM streptavidin forms were incubated with 200 μM biotin. Less than 2 min before introducing into the mass spectrometer, the buffer-exchanged samples were diluted with a solution of 1:1 15 mM ammonium acetate pH 7.8 and 78% acetonitrile, 0.01% trifluoroacetic acid.

An Advion nanospray robot (Advion BioSystems, Ithica, N.Y.) with a back-pressure of 0.45 Psi introduced the samples into the mass spectrometer, an 8.5 Tesla custom-built Electrospray Ionisation-Fourier Transform Mass Spectrometer. To visualize the non-covalent tetramers and non-covalent biotin binding in the high m/z range, the following settings were used: Chirp rate=750 Hz, Amplitude=0.5 V p-p, Tube lens=200 V, Capillary heater 2 V, Quad filter=−20 V, Skimmer=0 V, Capillary offset=34 V, X-fer=−110 V, Leak gas=4.2×10⁻⁵ Torr. The capillary heater was kept low and the Quad filter and Skimmer were kept either high or off to prevent subunit dissociation. The transfer was set to this low value of −110 V in order to visualize the high m/z region.

The masses were calculated manually by first determining the charge state. The final mass was determined by multiplying the observed m/z by the charge and subtracting the mass corresponding to the addition of protons to give that charge. For example, for the 15+charge state of D4 an m/z of 3534.159 was obtained. (15×3534.159)-(15×1.00727) gave a mass for this ion of 52,997 Da. This calculation was repeated for each charge state and the mean and standard deviation reported. The spectra were calibrated with tetrameric streptavidin, after its monomer mass was determined under denaturing conditions (Mr=13,271.4 Da). Average masses were predicted from the DNA sequence, using the ExPASy PeptideMass Calculator (ca.expasy.org/tools/peptide-mass.html) and assuming removal of the N-terminal formyl-Methionine.

K_{d} Measurements

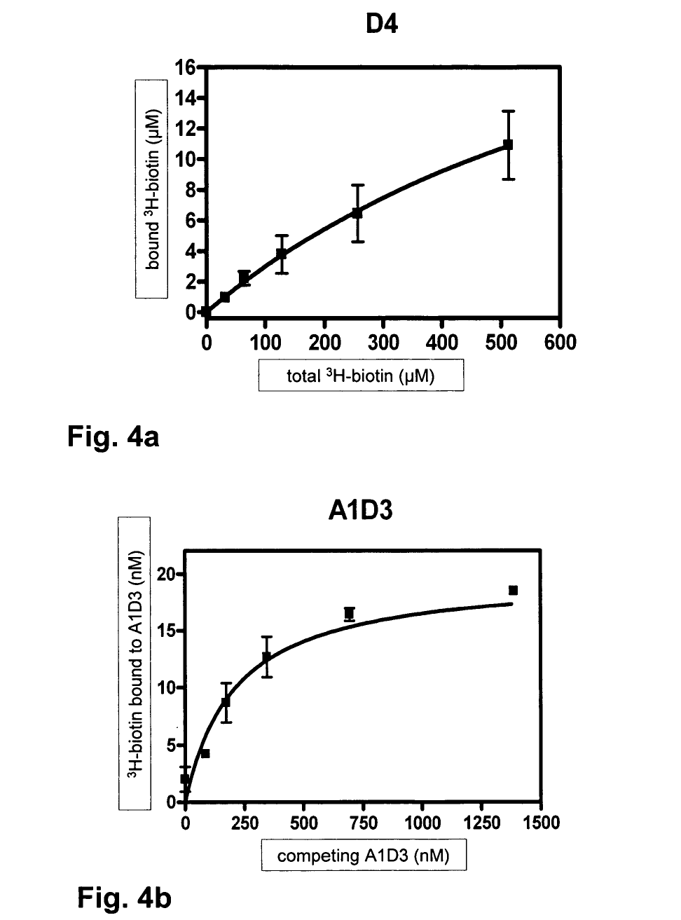

The K_{d} of A1D3 streptavidin (A is “Alive” subunit; D is “Dead” subunit) was obtained using a competition assay modified from Klumb et al. (Klumb, L. A. et al., Biochemistry 37: 7657-7663, 1998). Wild-type streptavidin was depleted of the small amount of co-purifying monomeric streptavidin by gel filtration. Fully tetrameric wild-type streptavidin (60 nM each subunit) was mixed with 20 nM (Kada, G. et al., Biochim. Biophys. Acta 1427: 33-43, 1999; Bayer, E. A. et al., Electrophoresis 17: 1319-1324, 1996) ³H-biotin (Amersham; Piscataway, N.J.) and 0-1.4 μM of competing A1D3 in PBS pH 7.0. Mixtures were incubated at 37° C. for >20 hr to allow sufficient time for equilibration. To separate the His₆-tagged A1D3 from wild-type streptavidin, an equal volume of a 50% slurry of Ni-NTA beads (Qiagen) in PBS with 15 mM imidazole was added. After 1 hr at room temperature, the beads were cleared by centrifugation at 15,600 g for 1 min. Aliquots were taken from the supernatant containing the biotin-bound wild-type streptavidin, an equal volume of 10% SDS in water was added, and samples were heated to 95° C. for 30 min, and counted in a Beckman Coulter LS6500 Liquid Scintillation Counter. The K_{d} ratio was obtained using Matlab (Mathworks; Natick, Mass.) using the formula from Klumb et al. (Klumb, L. A. et al., Biochemistry 37: 7657-7663, 1998). The affinity of A1D3 was calculated from this K_{d} ratio multiplied by the previously determined K_{d} of wild-type streptavidin for biotin of 4×10⁻¹⁴M (Green, N. M. Methods Enzymol. 184: 51-67, 1990) and divided by four, since only one of the four subunits of A1D3 showed significant biotin binding (FIG. 4A).

It was difficult to detect biotin binding by D4 using a competition assay against wild-type streptavidin because of its extremely low binding affinity, and so the following assay was used instead to determine the K_{d} (Reznik, G. O. et al., Proc. Natl. Acad. Sci. USA 95: 13525-13530, 1998): 24 μM D4 was incubated with 0-500 μM ³H-biotin in 100 μL total volume. After incubation at room temperature for 20 hr, the protein was precipitated by adding 50 μL to 200 μL 0.2 M ZnSO₄ followed by 200 μL 0.2 M NaOH. The protein precipitate was pelleted by centrifugation at 16,500 g for 5 min. The biotin bound by D4 was calculated from the total ³H-biotin added minus the ³H-biotin in the supernatant. The K_{d} was obtained using a nonlinear regression analysis (one-site binding hyperbola) with SigmaPlot (Systat Software; Point Richmond, Calif.).

Off-Rate Assay

The off-rate of biotin-fluorescein from streptavidin was measured in PBS with 20 mM HEPES pH 7.4 (PBS-H) using a Safire plate-reader and XFluor4 software (Tecan US; Durham, N.C.) with 494 nm excitation and 527 nm emission. In this assay the binding of biotin-4-fluorescein to an excess of streptavidin results in quenching of fluorescein emission (Kada, G. et al., Biochim. Biophys. Acta 1427: 33-43, 1999). As the biotin-4-fluorescein dissociates, the fluorescence recovers. The assay was performed in the presence of excess biotin so that sites left open by biotin-4-fluorescein dissociation are immediately re-filled by biotin. Streptavidin tetramer at 1 μM in 10 μL PBS-H was added to 12 nM biotin-4-fluorescein (Molecular Probes) in 170 μL PBS-H and incubated for 30 min at 37° C. 20 μL PBS-H or 20 μL PBS-H 10 mM biotin was then added and recording immediately started, with incubation at 37° C. Percentage dissociation was calculated as (signal with biotin−signal without biotin)/(mean maximal signal of T90I with biotin−initial T90I signal without biotin)×100. The concentration of competing biotin was saturating, since reducing the biotin concentration ten-fold produced indistinguishable dissociation rates.

Thermostability Assay

2.3 μM wild-type streptavidin or chimeric streptavidin in PBS was heated at the indicated temperature for 3 min in a PTC-200 PCR machine (MJ Research; Waltham, Mass.) and then immediately placed on ice (Bayer, E. A. et al., Electrophoresis 17: 1319-1324, 1996). Samples were mixed with 6×SDS-PAGE loading buffer and loaded onto a 16% polyacrylamide gel.

Cell Culture, Biotinylation and Imaging

HeLa cells were grown in Dulbecco's Modified Eagle Medium (DMEM) with 10% Fetal Calf Serum, 50 U/mL penicillin and 50 μg/mL streptomycin. HeLa stably expressing AP-CFP-TM or Ala-CFP-TM have been previously described (Howarth, M. et al., Proc. Natl. Acad. Sci. USA 102: 7583-7588, 2005). Dissociated primary neuronal cultures were prepared from Embryonic Day 18 or 19 (E18/19) rats and transfected with Lipofectamine 2000 at DIV6 as in Levinson et al. (Levinson, J. N. et al., J. Biol. Chem. 280: 17312-17319, 2005).

Enzymatic biotinylation and imaging of HeLa transfectants were performed as previously described (Howarth, M. et al., Proc. Natl. Acad. Sci. USA 102: 7583-7588, 2005), except instead of 10 μM biotin and 1 mM ATP, we added 10 μM biotin-AMP (synthesized according to Coleman and Huang; Coleman, T. M. and Huang, F. Chem. Biol. 9: 1227-1236, 2002) to give equivalent biotinylation but minimizing the risk of purinoreceptor activation by ATP (Rathbone, M. P. et al., Prog. Neurobiol. 59: 663-690, 1999). HeLa transfectants were biotinylated for 10 min at room temperature, and stained with 10 μg/mL Alexa Fluor 568-conjugated wild-type streptavidin, D4 or A1D3 for 10 min at 4° C. Biotinylation of neurons was performed at day in vitro (DIV) 8 in Hanks' Balanced Salt Solution (HBSS) (Invitrogen) with 0.2 μM biotin ligase and 10 μM biotin-AMP for 5 min at 37° C. Neurons were then washed with HBSS and incubated for 2 min with 5 μg/mL Alexa Fluor 568-conjugated wild-type streptavidin (Molecular Probes) or A1D3 at 37° C. Neurons were washed with NeuroBasal media (Invitrogen) supplemented with B-27 (Invitrogen), 50 U/mL penicillin, 50 μg/mL streptomycin, and 0.2 mM L-glutamine and chased in the same medium for 0 or 2 hr at 37° C. Cells were then fixed in −20° C. methanol. There was no signal from wild-type streptavidin labeling if neurons were instead transfected with Ala-neuroligin-1 containing a point mutation in AP, confirming the specificity of labeling (Howarth, M. et al., Proc. Natl. Acad. Sci. USA 102: 7583-7588, 2005). To observe synapse formation, cells were biotinylated and stained with streptavidin as above, biotinylation and streptavidin staining was repeated at 6 hr, and then after 24 hr total chase cells were fixed in methanol. Samples were stained for pre-synaptic markers using guinea pig anti-VGLUT1 (1:1000, Chemicon; Temecula, Calif.), followed by goat anti-guinea pig Alexa Fluor 488 (1:1000, Molecular Probes). All antibody reactions were performed in blocking solution [PBS with 0.3% Triton X-100 and 2% normal goat serum (Vector Laboratories; Burlingame, Calif.)] for 1 hr at room temperature or overnight at 4° C.

Images of HeLa cells were collected on a Zeiss Axiovert 200M inverted epifluorescence microscope using a 40× oil-immersion lens and a MicroMAX CCD camera (Roper Scientific; Tucson, Ariz.). CFP (420DF20 excitation, 450DRLP dichroic, 475DF40 emission) and Alexa568 (560DF20 excitation, 585DRLP dichroic, 605DF30 emission) images were collected and analyzed using OpenLab software (Improvision; Lexington, Mass.). Fluorescence images were background-corrected. Neuron images were acquired on a Zeiss Axiovert 200M microscope with a 63×1.4 NA Acromat oil-immersion lens and a monochrome 14-bit Zeiss Axiocam HR charged-coupled camera with 1300×1030 pixels. To correct for out-of-focus clusters within the field of view, focal plane z-stacks were acquired and maximum intensity projections performed off-line. Images were scaled to 16 bits and analyzed in Northern Eclipse (Empix Imaging; Ontario, Canada) with user-written software. Briefly, images were processed at a constant threshold level (of 32,000 pixel values) to create a binary mask image, which was multiplied with the original image using Boolean image arithmetic. The resulting image contained a discrete number of clusters with pixel values of the original image. Only dendritic clusters greater than 5 pixels in size, and with an average pixel values 2 times greater than background pixel values were used for analysis. Results were then calculated in terms of clusters per micrometer of dendrite. For assessment of pre-synaptic terminals, clusters were determined as before and average grey levels of clusters were compared between transfected dendrites and untransfected dendrites within the same field of view. The two-tailed parametric Student's t-test was performed to calculate statistical significance of results between experimental groups. “n” represents the number of transfected neurons for which clusters were measured.

### Example 5

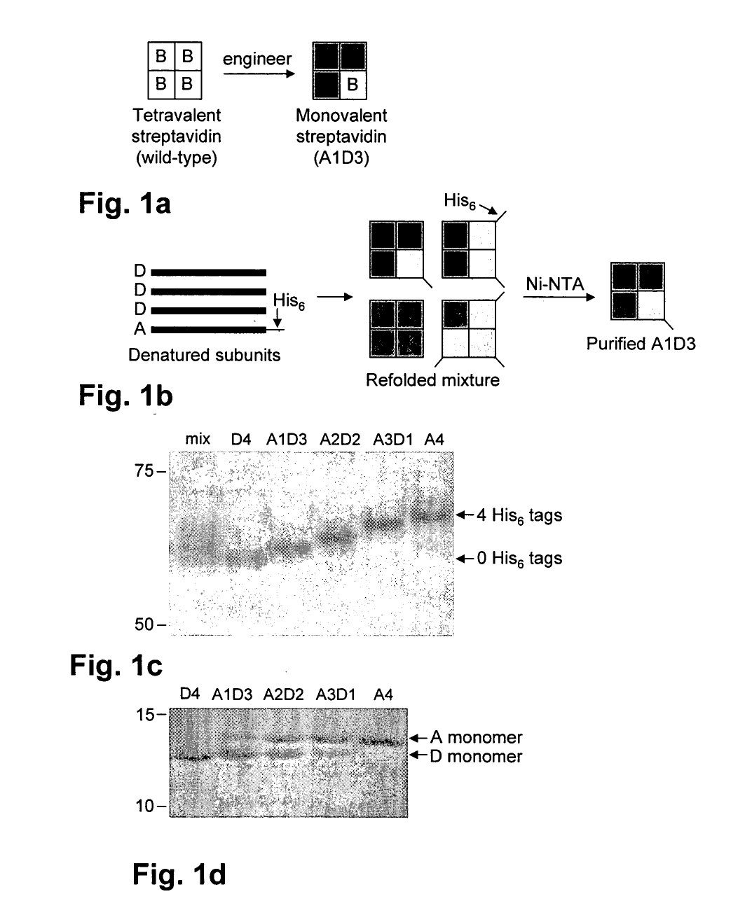

The strategy used to generate monovalent streptavidin is shown is FIG. 1A. Methods provided in the Methods section above were used to make and test the monovalent streptavidin. We wished to produce a streptavidin tetramer consisting of three subunits unable to bind biotin and one subunit that binds biotin as well as wild-type streptavidin. Many of the known mutations of streptavidin reduce biotin binding affinity dramatically (Qureshi, M. H. et al., J Biol. Chem. 276: 46422-46428, 2001; Chilkoti, A. et al., Proc. Natl. Acad. Sci. USA 92: 1754-1758, 1995; Klumb, L. A. et al., Biochem. 37: 7657-7663, 1998) but still leave K_{d} values in the nanomolar range and disrupt tetramerization (Qureshi, M. H. et al., J Biol. Chem. 276: 46422-46428, 2001; Wu, S. C. and Wong, S. L., J. Biol. Chem. 280: 23225-23231, 2005). The double mutant N23A, S27D has one of the weakest reported affinities for biotin (K_{d} 7.1×10⁻⁵ M¹⁰) and is still a tetramer. Nevertheless we observed that N23A, S27D streptavidin still bound to biotinylated cells. A triple mutant N23A, S27D, S45A was produced. The triple mutant N23A, S27D, S45A showed negligible biotin binding and left the tetramer structure intact. The binding of this triple mutant (composed of “Dead” subunits-D in FIG. 1) was so weak that it was difficult to measure but a K_{d} of 9.18±1.17×10⁻⁴ M (s.e.m.) was obtained. (FIG. 4A). To generate monovalent streptavidin (FIG. 1B), the wild-type subunit was first tagged with a His₆-tag (“Alive” subunit-A in FIG. 1). Then D and A subunits were combined at a molar ratio of 3:1 in guanidinium hydrochloride and refolded by rapidly diluting the mixture into PBS. This refold generated a mix of tetramers of different compositions.

The different tetramers were purified using a Ni-nitrilotriacetic acid (NTA) column, eluting according to the number of His₆-tags with increasing concentrations of imidazole. The tetramers could be distinguished by SDS-PAGE, if the samples were not boiled, according to the number of His-tags present, showing that at least 30% were of the monovalent A1D3 form (FIG. 1C, lanes 1 and 3). Thus purified fractions of the monovalent A1D3 were obtained (final yield 2 mg/L), as well as the other chimeric streptavidins, A2D2 and A3D1. The tetramer composition was further confirmed by boiling the samples before loading on SDS-PAGE, to determine the ratio of A to D subunits (FIG. 1D), and by electrospray ionization mass spectrometry. In Table 1 (Schwartz, B. L. et al., J. of the Amer. Soc. for Mass Spec. 6: 459-465, 1995) the observed mass (±s.d.), determined by Electrospray Ionization-Mass Spectrometry, is compared to the mass predicted from the sequence. From the change (±s.e.m.) upon addition of biotin (mass 244.31), we determined how many biotin molecules were bound to each tetramer.

TABLE 1
Mass of different streptavidin tetramers with or without biotin.

| | | | | | # |
|---|---|---|---|---|---|
| Tetramer | Predicted | Observed −biotin | Observed +biotin | Change +biotin | biotins |
| D4 | 52,962 | 52,997 ± 4 | 52,996 ± 12 | −1 ± 2 | 0 |
| A1D3 | 53,816 | 53,848 ± 5 | 54,088 ± 6 | 240 ± 4 | 1 |
| A2D2 | 54,669 | 54,704 ± 6 | 55,193 ± 3 | 489 ± 4 | 2 |
| A3D1 | 55,523 | 55,490 ± 45 | 56,201 ± 13 | 711 ± 39 | 3 |
| A4 | 56,377 | 56,394 ± 8 | 57,378 ± 8 | 984 ± 7 | 4 |

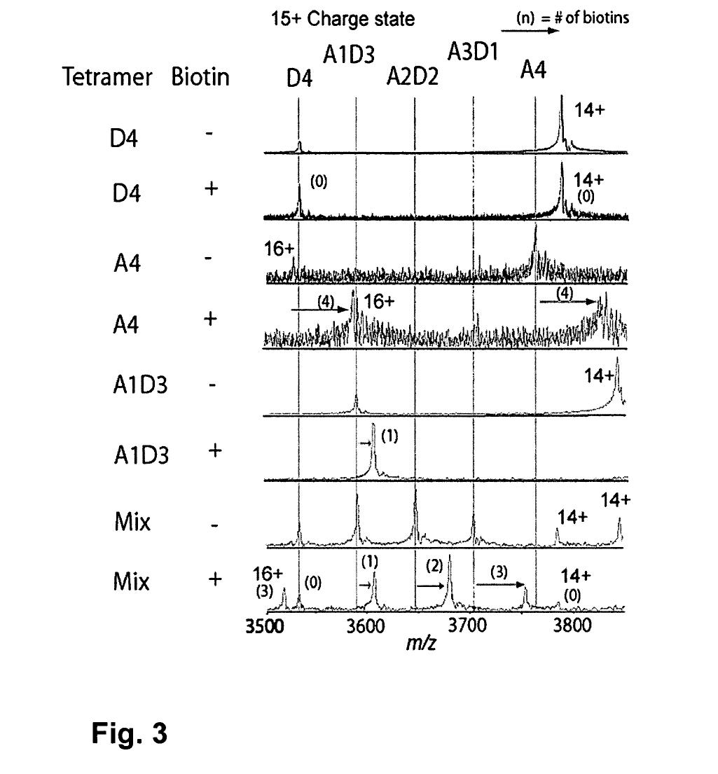

Despite the large mass of the streptavidin tetramer and non-covalent interaction between subunits, good agreement was found between expected and observed masses for D4, A1D3, A2D2, A3D1 and A4 (Table 1 and FIG. 3).

### Example 6

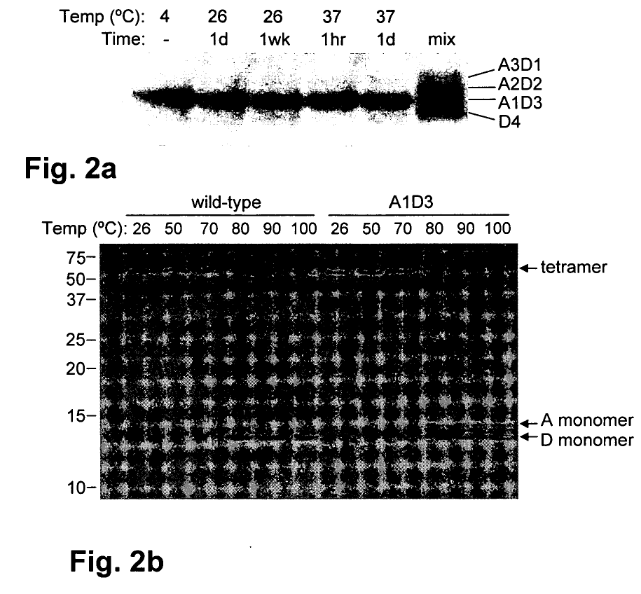

Methods described in the Methods section above herein were used for the following production and testing of monovalent streptavidin. Tests were performed to determine whether monovalent streptavidin would rearrange its subunit composition over time. A1D3 was incubated at room temperature or at 37° C. and analyzed by SDS-PAGE, to look for the appearance of D4 and A2D2 from subunit exchange (FIG. 2A). 2% of the A1D3 rearranged into D4 after 37° C. incubation for one day and 3% rearranged after room temperature incubation for one week. Formation of A2D2 was not detected in either case, indicating that significant fractions of multivalent streptavidin will not be generated upon storage. Next the stability of A1D3 to dissociation into monomers was tested, since many mutations in the biotin binding site of streptavidin weaken tetramer stability (Qureshi, M. H. et al., J Biol. Chem. 276: 46422-46428, 2001; Wu, S. C. and Wong, S. L., J. Biol. Chem. 280: 23225-23231, 2005). Wild-type streptavidin and A1D3 were heated in PBS at various temperatures and tetramer disassembly was determined by SDS-PAGE (FIG. 2B). A significant fraction of A1D3 remained tetrameric even at 100° C. There was little difference in thermostability between wild-type and monovalent streptavidin, suggesting that the mutations in D have minimal effect on the subunit interfaces and that it should be possible to use A1D3 in assays requiring high temperatures.

Electrospray ionization mass spectrometry was used to characterize the number of biotin molecules bound per tetramer. Spectra of the different streptavidin tetramers with or without biotin were acquired. As expected, all four subunits of A4 were associated with biotin (Table 1 and FIG. 3). No biotin binding by D4 could be detected. A1D3 was monovalent, binding a single biotin. The other chimeric tetramers bound one biotin per A subunit.

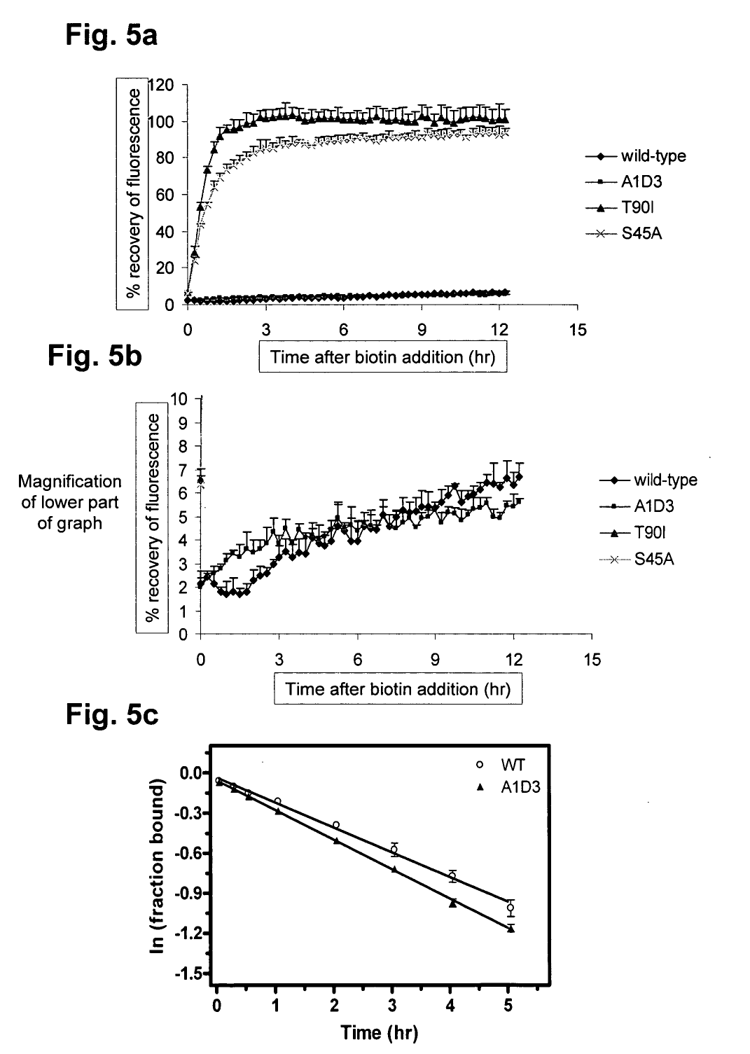

The biotin binding affinity of A1D3 was determined by measuring competition with wild-type streptavidin for ³H biotin (Klumb, L. A. et al., Biochem. 37: 7657-7663, 1998) (FIG. 4B). This indicated that the active biotin binding site in A1D3 has an affinity of 4.9±0.7×10⁻¹⁴ M (s.e.m.), based on the affinity of wild-type streptavidin of 4.0×10⁻¹⁴ M (Green, N. M. Methods in Enzymol. 184: 51-67, 1990). The stability of biotin-conjugate binding to A1D3 was also evaluated (FIG. 5). A previously characterized streptavidin mutant with a fast off-rate, S45A (Hyre, D. E. et al., Protein Sci. 9: 878-885, 2000), and a streptavidin mutant that was found to have a fast off-rate, T901, were used as a positive control for biotin-conjugate dissociation. S45A and T90I streptavidin showed >50% dissociation in 1 hour, whereas wild-type and A1D3 both dissociated less than 10% in 12 hours at 37° C.

To determine the off-rate of biotin from A1D3, 10 nM ³H-biotin was pre-incubated with 1 μM A1D3 or wild-type streptavidin for 20 minutes at 37° C. (Green, N. M. Methods in Enzymol. 184: 51-67, 1990). Dissociation was then initiated by addition of cold biotin at a final concentration of 50 μM and time-points taken over 5 hours at 37° C. 50 μL aliquots were removed and added to 200 μL 0.2M ZnSO₄ chilled on ice, followed by 200 μL 0.2 M NaOH. The protein precipitate was pelleted by centrifugation at 16,500 g for 5 min, and ³H-biotin in the supernatant was measured by liquid scintillation counting. Data were plotted as ln(fraction bound) versus time, and fit to a straight line by linear regression. Dissociation rates were deduced from the slope of the line and the equation:
ln(fraction bound)=−k_{off}(t)
where fraction bound=(total ³H-biotin−free ³H-biotin at timepoint)/(total ³H-biotin−free ³H-biotin before cold biotin chase). Results are shown in FIG. 5C. The measured off-rates were 5.17±0.25×10⁻⁵ s⁻¹ (s.e.m.) for wild-type streptavidin and 6.14±0.19×10⁻⁵ s⁻¹ (s.e.m.) for A1D3.

Site-specific biotinylation was used to study cell surface protein trafficking (Howarth, M. et al., Proc. Natl. Acad. Sci. USA 102: 7583-7588, 2005). Proteins of interest were tagged with a 15 amino acid acceptor peptide (AP), which was biotinylated by incubating cells with biotin ligase (BirA). The biotinylated protein was then tracked by labeling with fluorophore- or quantum dot-conjugated streptavidin (Howarth, M. et al., Proc. Natl. Acad. Sci. USA 102: 7583-7588, 2005), resulting in an interaction that was stable for many hours, unlike antibody labeling or other non-covalent site-specific labeling methods (Chen, I. and Ting, A. Y. Curr. Opin. Biotechnol. 16: 35-40, 2005).

Labeling of site-specifically biotinylated cell surface proteins with monovalent streptavidin was performed. Cyan fluorescent protein was tagged with AP and targeted to the surface of HeLa cells with a transmembrane domain (AP-CFP-TM). CFP-TM is cyan fluorescent protein with an acceptor peptide (AP), targeted to the cell surface with the transmembrane helix of PDGF receptor. HeLa expressing AP-CFP-TM or Ala-CFP-TM (a control with an alanine point mutation in AP) were biotinylated with biotin ligase for 10 min and stained with wild-type or monovalent (A1D3) streptavidin conjugated to Alexa Fluor 568. The Alexa-labeled and CFP-labeled images were overlaid. The results indicated no Alexa staining of AP-CFP-TM was observed when D4 was used or when biotin ligase was omitted and the cells were labeled with A1D3. Thus, after brief incubation with biotin ligase, biotinylated AP-CFP-TM was detected equally well with wild-type streptavidin or A1D3. However, an equivalent dye-conjugate of D4 gave no detectable staining, indicating that binding of A1D3 should only be through the A subunit. A point mutation in the acceptor peptide (Ala-CFP-TM) that blocked biotin ligase recognition abolished all staining. Staining was also abolished by omission of biotin ligase. Thus monovalent streptavidin did not give increased background in cell staining experiments compared to wild-type streptavidin.

### Example 7

Cross-linking is a central method of control of signal transduction, for example in the activation of growth factor receptors and transcription factors (Klemm, J. D. et al., Annu. Rev. Immunol. 16: 569-592, 1998), but is a concern when labeling cells with antibodies. Although Fab antibody fragments could be used to avoid cross-linking, Fabs are rarely of high affinity (making it difficult to label low abundance antigens) and will tend to dissociate on the time-scale of minutes. Cross-linking is disastrous for single-particle tracking experiments because the presence of an extra anchor slows protein diffusion (Iino, R. et al., Biophys J. 80: 2667-2677, 2001). It is normally said that using a ligand in excess will minimize cross-linking. However, labeled ligand must be present at a density of <1 per μm² for individual particles to be resolved. Thus these two requirements are only compatible if one is studying a target protein present at very low levels. There is still a need for a way to label surface proteins with an interaction of high stability that does not cross-link.

Methods described in the Methods section above were used for the following neuroligin tests. Neuroligins are post-synaptic adhesion proteins that play a role in the development of excitatory and inhibitory synapses (Scheiffele, P. et al., Cell 101: 657-669, 2000; Levinson, J. N. et al., J. Biol. Chem. 280: 17312-17319, 2005). Clustering of neuroligin has been observed during synapse development, but neuroligin's role in synapse initiation versus synapse stabilization is not clear.

To examine the effect of artificially-induced neuroligin clustering, AP-neuroligin-1 was site-specifically biotinylated at the cell surface with biotin ligase, and detected with either wild-type or monovalent streptavidin. Hippocampal neurons were transfected with AP-neuroligin-1, biotinylated with biotin ligase, and labeled with Alexa Fluor 568-conjugated wild-type streptavidin or A1D3. Cells were incubated for 0 or 2 hr at 37° C. and Alexa staining was visualized by fluorescence microscopy. Neurons were biotinylated and labeled with wild-type streptavidin or A1D3 as above, but incubated for 24 hr and then stained for the pre-synaptic marker VGLUT1. Streptavidin and VGLUT1 signals were assessed and their images overlaid for comparison. It was determined that the AP-neuroligin-1 clusters were not apposed to pre-synaptic terminals. At zero hours, diffuse surface staining of AP-neuroligin-1 was observed with both wild-type and monovalent streptavidin. After a two hour incubation, however, monovalent streptavidin-labeled AP-neuroligin-1 was still predominantly diffuse [clusters/μm 0.087±0.021 (s.e.m), n=9], but wild-type streptavidin-labeled AP-neuroligin-1 had formed distinct aggregates [clusters/μm 0.266±0.011 (s.e.m), n=9, p<0.0001], consistent with tetramer-induced protein cross-linking. The same staining pattern was observed after 24 hour incubation.

The aggregation of AP-neuroligin-1 by wild-type streptavidin correlated with reduced formation of excitatory pre-synaptic contacts, determined by the intensity of vesicular glutamate transporter-1 clusters (VGLUT1) (fold-enhancement of VGLUT1 cluster intensity: wild-type 1.71±0.07, monovalent 2.15±0.12, n=17, p<0.01), and by the fact that many of the aggregates induced by wild-type streptavidin were not apposed by pre-synaptic terminals positive for VGLUT1. Thus induction of neuroligin clustering by wild-type streptavidin had a deleterious effect on pre-synaptic differentiation. The increase in VGLUT1 cluster intensity for neurons transfected with AP-neuroligin-1 and labeled with monovalent streptavidin was similar to the increase seen for HA-neuroligin-1 transfected neurons (Prange, O. et al., Proc. Natl. Acad. Sci. USA 101: 13915-13920, 2004), suggesting that AP, biotin, and monovalent streptavidin did not disrupt the function of neuroligin-1 or neuroligin-neurexin interactions. Taken together with previous observations (Levinson, J. N. et al., J. Biol. Chem. 280: 17312-17319, 2005; Graf, E. R. et al., Cell 119: 1013-1026, 2004), these results suggest that while gradual neuroligin clustering from DIV7 to DIV14 may promote pre-synaptic differentiation, rapid clustering does not. These results also indicate that monovalent streptavidin can efficiently label proteins on the neuron surface, while avoiding the complications of aggregation of its target.

### Example 8

The need to purify different chimeric forms of streptavidin (Reznik, G. O. et al., Nat. Biotechnol. 14: 1007-1011, 1996; Chilkoti, A. et al., Biotechnology 13: 1198-1204, 1995) could be avoided if the four subunits could be genetically joined to make a single-chain streptavidin. However, the distance between the termini means that long linkers would be required, which are likely to impair folding. Attempts to circumvent this problem by circularly permuting streptavidin have yielded forms with K_{d}>10⁻⁸M (Chu, V. et al., Protein Sci. 7: 848-859, 1998; Aslan, F. M. et al., Proc. Natl. Acad. Sci. USA 102: 8507-8512, 2005). A circularly permuted tetravalent single-chain avidin with wild-type binding affinity was very recently generated (Nordlund, H. R. et al., Biochem., 2005). However, the ability to inactivate individual binding sites in this single-chain avidin has not been demonstrated and avidin binds less tightly to biotin conjugates than streptavidin (Pazy, Y. et al., J. Biol. Chem. 277: 30892-30900, 2002).

Tetravalent single chain avidin has been produced by Nordlund, et al., Biochemical Journal, published online, Aug. 10, 2005. Circularly permuted streptavidin is produced by joining the nucleotide sequences of streptavidin monomer subunits with linkers for expression as a single polypeptide chain. Wild-type streptavidin monomer subunit sequences are used to produce a single-chain produced wild-type streptavidin tetramer. The wild-type single chain-produced streptavidin has four wild-type binding domains.

A monovalent single chain-produced streptavidin tetramer is also produced. To make the monovalent single chain-produced streptavidin tetramer, the nucleotide sequence that encodes one wild-type streptavidin monomer subunit and the three copies of the nucleotide sequence that encodes a modified streptavidin monomer subunit are linked and a circularly permuted streptavidin is produced. The monovalent single chain-produced streptavidin tetramer has a binding affinity of wild-type streptavidin tetramer. The modified streptavidin subunits each have a sequence that includes at least three substituted amino acids. The substituted amino acids are N23A, S27D, and S45A. The modified streptavidin monomers do not functionally bind biotin when part of the monovalent single chain-produced streptavidin tetramer.

To make streptavidin dimer or trimer molecules, the nucleotide sequences of two or three streptavidin monomer subunits (respectively) are joined by linkers and a circularly permuted streptavidin dimer or trimer molecule is produced. The streptavidin monomer subunits are either wild-type or modified streptavidin monomer subunits. A modified streptavidin subunit includes the three substituted amino acids N23A, S27D, and S45A.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

We claim:

1. A monovalent avidin tetramer comprising three modified avidin monomer subunits and one wild-type avidin monomer subunit, wherein the modified avidin monomer subunits each bind biotin or a fragment thereof with a K_{d} of greater than or equal to about 1×10⁻⁴ M.

2. The monovalent avidin tetramer of claim 1, wherein the wild-type avidin monomer subunit binds biotin or a fragment thereof, with a K_{d} of a wild-type avidin monomer subunit for binding biotin or a fragment thereof.

3. The monovalent avidin tetramer of claim 1, wherein the monovalent avidin tetramer has a proximal avidin K_{d} for binding biotin or a fragment thereof.

4. The monovalent avidin tetramer of claim 1, wherein the monovalent avidin tetramer has a single femtomolar biotin binding site.

5. The monovalent avidin tetramer of claim 1, wherein the monovalent avidin tetramer has a proximal avidin overall biotin off rate.

6. The monovalent avidin tetramer of claim 1, wherein the amino acid sequence of the modified avidin monomer subunit consists of the amino acid sequence of a wild-type avidin monomer subunit with at least three substituted amino acid residues.

7. The monovalent avidin tetramer of claim 6, wherein the substituted amino acid residues are in the sequence of a biotin binding pocket of the avidin monomer subunit.

8. The monovalent avidin tetramer of claim 6, wherein the three substituted amino acid residues are N12A, S16D, and T35A.

9. The monovalent avidin tetramer of claim 1, wherein one or more of the modified or wild-type avidin monomer subunits further comprises a purification tag.

10. The monovalent avidin tetramer of claim 9, wherein the purification tag is a polyhistidine tag.

11. The monovalent avidin tetramer of claim 9, wherein the avidin monomer subunit further comprising the purification tag is the wild-type avidin monomer subunit.

12. The monovalent avidin tetramer of claim 1, further comprising a detectable label.

13. The monovalent avidin tetramer of claim 1, wherein the monovalent avidin tetramer is made by mixing together avidin monomers under conditions in which the monomers associate into tetramers.

14. The monovalent avidin tetramer of claim 1, wherein the monovalent avidin tetramer is made using a single-chain tetramer production method.

15-58. (canceled)

59. An avidin tetramer comprising N=1, 2, or 3 modified avidin monomer subunits and 4 minus N wild-type avidin monomer subunits, wherein:
(a) each wild-type avidin monomer subunit binds biotin or a fragment thereof with a K_{d} of a wild-type avidin monomer subunit for binding biotin or a fragment thereof,
(b) each modified avidin monomer subunit binds biotin or a fragment thereof with a K_{d} of greater than or equal to about 1×10⁻⁴ M, and
(c) the avidin tetramer has a proximal avidin K_{d} for binding biotin or a fragment thereof.

60-71. (canceled)

72. A plurality of avidin tetramers, wherein the tetramers are avidin tetramers of claim 59 and the plurality includes avidin tetramers that have a ratio of wild-type avidin monomer subunits to modified avidin monomer subunits of 1:3, 2:2, 3:1, or a mixture thereof.

73. A method of making a plurality of avidin tetramers comprising 1, 2, or 3 modified avidin monomer subunits, wherein the tetramer is formed by associating wild-type avidin monomers with modified avidin monomers, wherein the avidin tetramers have one or more of the following characteristics:
(a) each wild-type avidin monomer subunit binds biotin or a fragment thereof a with a K_{d} of a wild-type avidin monomer subunit for binding biotin or a fragment thereof,
(b) the modified avidin monomer subunits bind biotin or a fragment thereof with. a K_{d} of greater than or equal to about 1×10⁻⁴ M, and
(c) the avidin tetramer has a proximal avidin K_{d} for binding biotin or a fragment thereof, and
(d) the tetramer has a proximal avidin K_{d} for binding biotin or a fragment thereof.

74-86. (canceled)

87. A method of binding biotin or a fragment thereof comprising:
contacting a biological sample comprising biotin or a fragment thereof with a monovalent avidin tetramer of claim 1 under conditions that permit binding of biotin or a fragment thereof with a monovalent avidin tetramer.

88. A method of binding biotin or a fragment thereof comprising:
contacting a biological sample comprising biotin or a fragment thereof with an avidin tetramer of claim 59 under conditions that permit binding of biotin or a fragment thereof with an avidin tetramer.

89-104. (canceled)

105. A method of binding biotin or a fragment thereof comprising:
contacting a biological sample comprising biotin or a fragment thereof with an avidin tetramer made by the method of claim 73 under conditions that permit binding of biotin or a fragment thereof with an avidin tetramer.
